(19) United States
(12) Patent Application Publication
(10) Pub. No.: US 2017/0326045 A1
(43) Pub. Date: Nov. 16, 2017

(54) COSMETIC COMPOSITION COMPRISING A SYNTHETIC PHYLLOSILICATE AND A POLYOL AND/OR A UV FILTER

(71) Applicant: L'OREAL, Paris (FR)

(72) Inventors: Raluca LORANT, Chevilly-Larue (FR); Mathieu CHABRILLANGEAS, Chevilly-Larue (FR); Chantal JOUY, Chevilly-Larue (FR); Marie-Lise CHIRON, Chevilly-Larue (FR)

(73) Assignee: L'OREAL, Paris (FR)

(21) Appl. No.: 15/529,431

(22) PCT Filed: Nov. 24, 2015
(86) PCT No.: PCT/EP2015/077549

§ 371 Date: May 24, 2017

(30) Foreign Application Priority Data
Nov. 24, 2014 (FR) 1461331
Nov. 24, 2014 (FR) 1461344

(51) Int. Cl. A61K 8/25; A61K 8/34; A61K 8/60; A61Q 17/04

(52) U.S. Cl. CPC A61K 8/25; A61Q 17/04; A61K 8/345; A61K 8/60

(57) ABSTRACT

The present invention relates to a composition, in particular cosmetic composition, comprising: (a) at least one synthetic phyllosilicate having the molecular formula Mg₃Si₄O₁₀(OH)₂; and (b) at least one polyol and/or polyol derivative, and/or at least one UV filter. The present invention further relates to the use of a synthetic phyllosilicate having the molecular formula Mg₃Si₄O₁₀(OH)₂ for reducing the tackiness and/or for increasing the hydrating effect of said composition, the cosmetic use of said composition for topical skin and/or nail care, a cosmetic treatment method comprising topical application of said composition to the skin and/or the nails, a cosmetic method for limiting the darkening of the skin and/or the nails and/or for improving the colour and/or the homogeneity of the complexion, and a cosmetic method for preventing and/or treating signs of ageing of the skin and/or the nails comprising applying said composition to the surface of the skin and/or the nails.

The present invention relates to compositions, in particular cosmetic compositions, such as emulsions, comprising at least one synthetic phyllosilicate and at least one polyol and/or polyol derivative and/or at least one UV-screening agent. When at least one UV-screening agent is present, the present invention is also directed toward photoprotective compositions, preferably photoprotective cosmetic or dermatological compositions, also called anti-sun products or photoprotective products, and more particularly of emulsion, gel or emulsified gel type. More particularly, it relates to the field of caring for, cleansing, protecting and/or making up the skin and/or nails, and in particular facial and/or bodily skin.

The term “skin” is intended to mean facial and/or bodily skin.

The term “nails” is also intended to mean false nails in so far as the cosmetic effects desired are often the same.

It is known that keratin materials such as the skin have a tendency to dry out owing to environmental factors (pollution, wind, cold, air-conditioning), psychological factors (fatigue, stress) or hormonal factors (menopause). However, it is important for the skin to be well hydrated and not to undergo any water loss which risks leading to weathering and drying out of the skin. Consumers thus expect their cosmetic products to moisturize their skin well.

The feeling of moisturized skin is conveyed, on application, by products that provide an aqueous sensation. An aqueous sensation is often associated with a persistent fresh effect provided by the composition. The expression “composition providing a persistent fresh effect” is intended to mean a composition which, after application, creates a reduction in perceptible skin temperature, for example of at least 0.5° C., this fresh effect lasting from application up to at least 4 minutes after application.

In the longer term, the feeling of moisturized skin is conveyed by a feeling of supple skin and of moisturized upper epidermal layers.

To provide this moisturization of the upper epidermal layers, it is common to incorporate humectants, which are hygroscopic substances that bring about rehydration of the skin by uptake of atmospheric water and by retention of water in the skin. Examples of these humectants are the constituents of NMF (Natural Moisturizing Factor), such as urea, or polyols, which includes sugars and glycols.

The higher the content of these humectants, the greater is their moisturizing efficiency.

Moreover, facial and/or bodily skin is exposed daily to sunlight.

It is known that radiation with wavelengths of between 280 nm and 400 nm makes possible tanning of the human epidermis and that radiation with wavelengths of between 280 nm and 320 nm, known under the name of UVB rays, harms the development of a natural tan. Exposure is also liable to induce impairment of the biomechanical properties of the epidermis, which is reflected by the appearance of wrinkles, leading to premature aging of the skin.

It is also known that UVA rays of wavelengths between 320 nm and 400 nm penetrate more deeply into the skin than UVB rays. UVA rays cause immediate and persistent browning of the skin. Daily exposure to UVA rays, even for a short period, under normal conditions may lead to degradation of the collagen and elastin fibers, which is reflected by a change in the skin's microrelief, the appearance of wrinkles and non-uniform pigmentation (liver spots, or heterogeneity of the complexion).

Many photoprotective compositions have been proposed to date to overcome the effects induced by UVA and/or UVB radiation.

To counter these harmful effects, it is common practice to use photoprotective compositions in which organic or inorganic UV-screening agents (or sunscreens) are formulated.

One of the major issues for photoprotective formulations of this type is to introduce UV-screening agents that are effective, that is to say that provide optimal UV protection at the desired protection factors, while at the same time providing a good level of sensoriality and of cosmetic pleasantness in order to prompt the use of anti-sun products and thus to protect the skin against attacks by ultraviolet rays.

In order to guarantee high sun protection factors, these UV-screening agents can be used in high concentrations, sometimes above 20% by weight relative to the total weight of the composition.

However, as set out hereinafter, these humectants and UV-screening agents have certain drawbacks, including that of affecting the sensory properties of the products by causing in particular unwanted effects.

Indeed, the introduction of these humectants, in particular polyols such as glycerine, into a physiologically acceptable medium can lead to a modification of the sensory properties of the products, by causing a tacky effect, and by reducing the aqueous sensation on application. The higher the polyol content, the greater is this sensory modification which causes an impression of a lack of moisturizing efficiency on application.

In addition, polyols, such as sugars and derivatives thereof, are capable of forming formulations which “slip” on application, making their penetration difficult, and which can produce a soaping effect on application. These drawbacks are in particular noticeable when the polyol is a sugar derivative such as an alkyl polyglucoside and/or a sucrose ester or a glucose ether, very often used as emulsifiers.

This soaping effect is all the greater when the composition does not contain a silicone-based fatty substance.

Finally, it should be noted that the combination, with these polyols, of certain active agents, in particular anti-aging active agents, can accentuate these modifications.

With regard to sunscreens, their use in cosmetic compositions degrades the sensory pleasantness of the support architectures. This is because, generally, UV-screening agents, depending on whether they are water-soluble or liposoluble, introduce various types of unpleasantness or discomfort such as a tacky, greasy, coarse or dragging effect on the skin, and a lack of freshness and of comfort.

In addition, the introduction of these sunscreens into emulsified and/or gelled galenical formulations brings about destabilization problems. This instability can even sometimes cause phase separation of the emulsion and/or a loss of viscosity of the composition, making the formulation inefficient or even unusable. There thus remains the need to provide compositions which provide a satisfactory feeling of moisturization on application, and real moisturizing efficiency on the skin after application.

There thus also remains the need to produce cosmetic compositions which are both non-tacky and aqueous on application and which contain at least one polyol, in particular a glycol such as glycerine.

There is in particular a need to produce cosmetic compositions which are both non-tacky and aqueous on application and which contain a high content of a polyol, in particular a glycol such as glycerine.

There also remains the need to prepare compositions which provide little or no wet effect on application, nor any tacky effect, which are devoid of a greasy sensation and the drying time of which after application to the skin and/or the nails is shortened compared with the compositions known in the cosmetics field.

There also remains the need to produce various types of architectures which provide a good level of sensory pleasantness, in particular in terms of effect of lightness or even of freshness with a substantial reduction in the greasy residue, in the tacky effect, in the coarse effect and/or in the shiny effect on the skin. The object of the present invention is in particular to meet this need while at the same time aiming to reinforce the efficiency of these compositions in terms of photoprotection.

The inventors have noted, surprisingly, that the use of a synthetic phyllosilicate in combination with these polyols and/or polyol derivatives and/or UV-screening agents makes it possible to obtain products which do not have a tacky effect, which do not “slip” and the whitening effect of which during or after application is reduced.

Thus, a subject of the present invention is a composition, in particular a cosmetic composition, comprising:
(a) at least one synthetic phyllosilicate of molecular formula Mg₃Si₄O₁₀(OH)₂; and
(b) at least one polyol, and/or polyol derivative, and/or at least one UV-screening agent.

According to one particular embodiment, a composition of the invention comprises:
(a) at least one synthetic phyllosilicate of molecular formula Mg₃Si₄O₁₀(OH)₂; and
(b) at least one polyol, and/or polyol derivative.

According to another particular embodiment, a composition of the invention comprises:
(a) at least one synthetic phyllosilicate of molecular formula Mg₃Si₄O₁₀(OH)₂; and
b) at least one UV-screening agent.

According to another particular embodiment, a composition of the invention comprises:
(a) at least one synthetic phyllosilicate of molecular formula Mg₃Si₄O₁₀(OH)₂;
(b) at least one polyol and/or polyol derivative;
(c) at least one aqueous phase; and
(d) at least one fatty phase.

Advantageously, the composition according to the present invention comprising said phyllosilicate presents an X-ray diffraction line greater than 9.4 Å and less or equal to 9.8 Å.

Advantageously, the composition according to the present invention comprising said phyllosilicate has an infrared absorption band at 7200 cm⁻¹, corresponding to the stretching vibration attributed to the silanol groups Si—OH at the edge of the phyllosilicate leaflets.

Advantageously, the composition according to the present invention comprising said phyllosilicate is characterized by the absence of an infrared absorption band at 7156 cm⁻¹. This band at 7156 cm⁻¹ corresponds to the vibration band of Mg₂FeOH.

The composition according to the present invention comprising said phyllosilicate also preferably has an infrared absorption band at 7184 cm⁻¹ corresponding to the 2 ν Mg₃OH stretching vibration.

It should be noted that in the presence of adsorbed water, for example residual water, a broad infrared absorption band is detectable and readily identifiable, for example at 5500 cm⁻¹.

According to a first variant, said synthetic phyllosilicate is used there in the form of an aqueous or aqueous-alcoholic gel.

According to a second variant, said synthetic phyllosilicate is used there in a dry (or powder) particulate form.

According to a third variant, said synthetic phyllosilicate is used there in the form of an aqueous or aqueous-alcoholic gel and in a dry (or powder) particulate form.

According to an embodiment, the composition according to the invention is a cosmetic or dermatological composition comprising a physiologically acceptable medium.

Synthetic phyllosilicates such as those described in application WO 2008/009799 and advantageously those disclosed in application FR 2 977 580 are most particularly suitable for use in the invention.

However, neither of these documents WO2008/009799 and FR 2 977 580 considers exploiting the resulting synthetic phyllosilicates in compositions, in particular, relating to cosmetic, dermatological or pharmaceutical applications.

In particular, neither of these documents considers a combination between these synthetic phyllosilicates and polyols or polyol derivatives and/or UV-screening agents.

As emerges from the examples hereinafter, the combination considered according to the invention is particularly advantageous for (i) reducing the tacky effect, and (ii) increasing the moisturizing effect of compositions, in particular cosmetic compositions, in particular compositions for topical application, comprising polyols and/or polyol derivatives.

Indeed, generally, the introduction of a synthetic phyllosilicate suitable for use in the invention, in gel and/or powder form, into a formula containing at least one emulsifier which is a polyol and/or a polyol derivative makes it possible to reduce the tacky feeling of the emulsion and to have a better feeling of penetration of the formula during application. The compositions according to the invention slip less than a placebo formula, that is to say a formula which does not comprise any synthetic phyllosilicate suitable for use in the invention.

The penetration sensation correlates directly with the moisturization sensation, and therefore with the perception of effectiveness of the product. The compositions according to the invention also have a clean-skin finish.

A subject of the invention is also the cosmetic use of a composition as defined above, for the topical care of the skin and/or the nails, in particular for the care of bodily and/or facial skin, and/or the nails.

According to another aspect, a subject of the invention is a cosmetic treatment process comprising the topical application to the skin and/or the nails of a composition of the invention.

According to another aspect, a subject of the invention is the use of a synthetic phyllosilicate of molecular formula Mg₃Si₄O₁₀(OH)₂ as defined hereinafter, in a composition comprising at least one polyol or a polyol derivative, in order to reduce the tacky effect of said composition.

According to another aspect, a subject of the invention is thus also the use of a synthetic phyllosilicate of molecular formula Mg₃Si₄O₁₀(OH)₂ as defined hereinafter, in a composition comprising at least one polyol or a polyol derivative, in order to increase the moisturizing effect of said composition.

The technical effect observed by implementing this combination is also particularly advantageous with regard to compositions comprising these said polyols and/or derivatives.

The technical effect observed by implementing this combination is also particularly advantageous with regard to compositions comprising high contents of said polyols and/or derivatives, and more particularly comprising a high content of glycols.

“High contents” of these polyols and/or derivatives, preferably of these glycols, including in particular compositions comprising at least 20% by weight, at least 30% by weight of said polyol(s), preferably of said glycols, relative to the total weight of the composition.

In addition, as emerges from the examples hereinafter, the inventors have noted that the combination of a synthetic phyllosilicate, in particular as defined below, described in FR 2 977 580, with at least one UV-screening agent as defined below makes it possible to obtain anti-UV compositions which have improved sensory qualities and a decrease in the tacky effect, in the greasy effect and in the coarse effect of the UV-screening agents, and even which have an improved SPF (Sun Protection Factor) performance level and/or even a stability.

For the purposes of the invention, the term “SPF” is intended to mean the sun protection factor, which measures the level of protection against UVB radiation. The value of the SPF corresponds to the ratio between the minimum time necessary to obtain sunburn with a sunscreen composition and the minimum time without product.

It is expressed mathematically by the ratio of the UV radiation dose necessary to reach the erythemogenic threshold with the UV-screening agent to the UV radiation dose necessary to reach the erythemogenic threshold without UV-screening agent. This factor thus concerns the efficacy of the protection having a spectrum of biological action mainly centered in the UVB range and consequently gives an account of the protection with regard to this UVB radiation.

It also relates to a cosmetic process for limiting the darkening of the skin and/or the nails and/or improving the color and/or uniformity of the complexion, comprising the application, to the surface of the skin and/or the nails, of at least one composition as defined previously, said composition comprising at least one UV-screening agent.

It is also directed toward a cosmetic process for preventing and/or treating the signs of aging of the skin and/or of the nails, comprising the application, to the surface of the skin and/or the nails, of at least one composition as defined previously, said composition comprising at least one UV-screening agent.

The composition according to the invention is suitable for topical application.

Synthetic Phyllosilicate

The synthetic phyllosilicate in accordance with the invention has a crystalline structure in accordance with that of a hydroxylated magnesium silicate of molecular formula Mg₃Si₄O₁₀(OH)₂ belonging to the chemical family of phyllosilicates.

These phyllosilicates are generally formed from a stack of elemental leaflets of crystalline structure, the number of which ranges from a few units to several tens of units. Each elemental leaflet is formed by the association of two layers of tetrahedra in which the silicon atoms are positioned, located on either side of a layer of octahedra in which the magnesium atoms are positioned. This group corresponds to the 2/1 phyllosilicates, which are also termed as being of T.O.T. (tetrahedron-octahedron-tetrahedron) type.

As presented above, a synthetic phyllosilicate in accordance with the invention may be obtained according to a preparation process such as the one described in application WO 2008/009799 and is preferentially obtained according to the technology described in application FR 2 977 580.

This preparation process in particular comprises a prolonged hydrothermal treatment, which makes it possible to obtain an aqueous gel of synthetic phyllosilicate. Accordingly, according to a first embodiment variant, the synthetic phyllosilicate may be used in the form of an aqueous or aqueous-alcoholic gel, in particular like the one obtained directly on conclusion of the synthesis process.

As described in application FR 2 977 580, the parameters that influence the synthesis and the properties of a synthetic phyllosilicate in gel form that is suitable for use in the invention are the nature of the heat treatment (200° C. to 900° C.), the pressure, the nature of the reagents and the proportions thereof.

More particularly, the duration and temperature of the hydrothermal treatment make it possible to control the size of the particles. For example, the lower the temperature, the smaller the synthesized particles, as described in application FR 2 977 580. Controlling the size makes it possible to afford new properties and better control of both its hydrophilic and hydrophobic properties, i.e. amphiphilic properties.

It should nevertheless be noted that the gel as obtained after the synthesis process may be subject to an optional washing step with water/centrifugation, after which it is dried and milled. The synthetic phyllosilicate is then available in the form of powder.

Accordingly, the synthetic phyllosilicate considered according to the invention may also be formulated in the state of powder within a composition according to the invention.

Structural Analysis and Characterization of a Synthetic Phyllosilicate that is Suitable for Use in the Invention

A synthetic phyllosilicate that is suitable for use in the invention may be characterized by various parameters, namely infrared absorption bands, its size and its purity, as detailed below.

Under certain conditions, analyses such as nuclear magnetic resonance in particular of ²⁹Si may be useful for the characterization of a synthetic phyllosilicate that is suitable for use in the invention. Similarly, thermogravimetric analysis (TGA) may be used for the characterization of a synthetic phyllosilicate that is suitable for use in the invention. Finally, x-ray diffraction may also be used for this purpose.

Infrared

Method Used

The machine used is a Nicolet 6700 FTIR Fourier transform spectrometer, equipped with an integration sphere, with an InGaA detector and a CaF₂ separator and a resolution of 12 cm⁻¹, more preferentially of 8 cm⁻¹ and even more preferentially of 4 cm⁻¹. In other words, the values of the infrared absorption bands given in this description should be considered as being approximately 6 cm⁻¹ and more preferentially approximately 4 cm⁻¹ and even more preferentially approximately 2 cm⁻¹.

The near infrared recordings of the stretching region located at 7184 cm⁻¹ were broken down by pseudo-Voigt functions using the Fityk software (Wojdyr, 2010).

To visualize the absorption spectrum in a composition comprising at least one aqueous part, such as an emulsion, it is recommended to heat this composition to a temperature corresponding to a temperature of greater than or equal to 100° C. (for example 120° C.) and less than or equal to 500° C. (for example 400° C.) so as to remove the adsorbed water part and, where appropriate, some or all of the organic compound(s) present in the composition.

Generally to confirm an infrared absorption band, the person skilled in the art performs stretching enlargements, specifically, he may for example enlarge by approximately 200 cm⁻¹ either side of a suspected infrared absorption band.

A natural talc is a mineral species composed of doubly hydroxylated magnesium silicate having formula Mg₃Si₄O₁₀(OH)₂, which may contain traces of nickel, iron, aluminum, calcium or sodium.

Natural talc has an infrared spectrum with a typical, fine and strong absorption band at 7184 cm⁻¹ corresponding to the 2 ν Mg₃OH stretching vibration. Natural talc also contains chemical elements which replace magnesium and silicon in the crystalline structure, which impose the appearance of at least one additional infrared absorption band, in particular that corresponding to the stretching vibration at 7156 cm⁻¹ attributable to 2 ν Mg₂FeOH.

The spectrum of the synthetic phyllosilicate that is suitable for use in the invention differs from that of a natural talc by an infrared absorption band at 7200 cm⁻¹ corresponding to the stretching vibration attributed to the silanol groups Si—OH at the edge of the phyllosilicate leaflets.

To confirm this infrared absorption band, those skilled in the art may perform a stretching amplification, in particular in the region 7400 cm⁻¹−7000 cm⁻¹ and more particularly in the region 7300 cm⁻¹−7100 cm⁻¹.

Preferably, the spectrum of the synthetic phyllosilicate is also characterized by an absence of infrared absorption band at 7156 cm⁻¹. This band at 7156 cm⁻¹ corresponds to the vibration band of Mg₂FeOH.

Preferably, the spectrum of the synthetic phyllosilicate is also characterized by the infrared absorption band at 7184 cm⁻¹ which is common to natural talc.

It should be noted that in the presence of adsorbed water, for example residual water, a broad infrared absorption band is detectable and readily identifiable, for example at 5500 cm⁻¹.

Advantageously, the composition according to the present invention comprising said phyllosilicate has an infrared absorption band at 7200 cm⁻¹, corresponding to the stretching vibration attributed to the silanol groups Si—OH at the edge of the phyllosilicate leaflets.

Advantageously, the composition according to the present invention comprising said phyllosilicate is characterized by the absence of an infrared absorption band at 7156 cm⁻¹. This band at 7156 cm⁻¹ corresponds to the vibration band of Mg₂FeOH.

The composition according to the present invention comprising said phyllosilicate also preferably has an infrared absorption band at 7184 cm⁻¹ corresponding to the 2 ν Mg₃OH stretching vibration.

In a composition according to the invention, it should be noted that in the presence of adsorbed water, for example residual water, a broad infrared absorption band is detectable and readily identifiable, for example at 5500 cm⁻¹.

Size

Method Used

To perform the particle size analysis of the synthetic phyllosilicates that are suitable for use in the invention, photon correlation spectroscopy was used. This analytical technique affords access to the size of the particles on the basis of the principle of dynamic light scattering. This device measures over time the intensity of the light scattered by the particles at an angle θ under consideration and the scattered rays are then processed using the Padé-Laplace algorithm.

This non-destructive technique requires dissolution of the particles. The particle size measurement obtained by this technique corresponds to the value of the hydrodynamic diameter of the particle, i.e. it comprises both the particle size and also the thickness of the hydration layer.

The analyses were performed using a VASC0-2 particle size analyzer from Cordouan. For the purpose of obtaining statistical information regarding the particle distribution, the NanoQ™ software was used in multi-acquisition mode with the Padé-Laplace algorithm.

Thus, a synthetic phyllosilicate that is suitable for use in the invention, in the form of an aqueous or aqueous-alcoholic gel, advantageously has a mean size ranging from 300 nm to 500 nm.

By contrast, a synthetic phyllosilicate when it is used in the form of a powder, in the image of that obtained by dehydration of an aqueous gel, as defined above, may have an average size ranging from a few microns to several hundred microns, preferably ranging from 5 μm to 100 μm, or may be presented in the form of porous micron or multimicron aggregates composed of said particles.

These characteristics are advantageous with regard to a natural talc, one of the constraints of which is the uncontrolled size of its particles.

Purity

The synthetic phyllosilicate under consideration according to the invention has a degree of purity of at least 99.90% and preferably of at least 99.99%.

It is thus advantageously free of impurities or of undesirable compounds, among which are in particular asbestos minerals such as asbestos (serpentine), chlorite, carbonates, heavy metals, iron sulfides, etc., which are generally associated with natural talc and/or incorporated into the structure of natural talcs.

NMR (Nuclear Magnetic Resonance)

Methods Used

The silicon-29 (²⁹Si) NMR spectra were recorded on a Bruker Avance 400 (9.4 T) spectrometer. The reference for the chemical shifts is tetramethylsilane (TMS). The samples were placed in 4 mm zirconia rotors. The magic angle spinning (MAS) speed was set at 8 kHz. The experiments were performed at the ambient temperature of 21° C.

The ²⁹Si spectra were obtained either by direct polarization (rotation of 30°) with a recycling delay of 60 seconds, or by cross polarization (CP) between 1H and ²⁹Si (recycling time of 5 seconds and contact time of 3 ms).

In silicon (²⁹Si) NMR, natural talc has a single peak at −97 ppm.

In silicon (²⁹Si) NMR, in contrast with natural talc, the spectrum of the synthetic phyllosilicate in accordance with the invention shows two peaks: one located at −95 ppm and the other located at −97 ppm, this being the case without the need for particle size fractionation to a size of less than 500 nm.

TGA (Thermogravimetric Analysis)

Method Used

The recordings were made using a Perkin Elmer Diamonds thermobalance.

For each analysis, about 20 mg of sample were required. During the analysis, the sample is subjected to a temperature increase ranging from 30° C. to 1200° C. at a rate of 10° C.·min⁻¹ under a stream of 100 mL·min⁻¹ of air.

The thermogravimetric analysis of a synthetic phyllosilicate in accordance with the invention shows a lower thermal stability (at about 800° C.) than that of natural talc and it is characterized by four losses of mass, in contrast with natural talc which has only one, at about 900° C.

To establish these losses of mass, it is useful to refer to the article by Angela Dumas, François Martin, Christophe Le Roux, Pierre Micoud, Sabine Petit, Eric Ferrage, Jocelyne Brendle, Olivier Grauby and Mike Greenhill-Hooper: “Phyllosilicates synthesis: a way of accessing edges contributions in NMR and FTIR spectroscopies. Example of synthetic talc” (Phys. Chem. Minerals, published on 27 Feb. 2013).

X-Ray Diffraction

Method Used

Analysis of the x-ray diffractogram, in particular with the aid of the material and method used for x-ray diffraction analyses, is detailed in application FR 2 977 580.

Preferably, given that x-ray diffraction is only performed on solids, to visualize the absorption spectrum in a composition comprising at least one aqueous part, such as an emulsion, it is recommended to heat this composition to a temperature corresponding to a temperature of greater than or equal to 100° C. (for example 120° C.) and less than or equal to 500° C. (for example 400° C.) so as to remove the adsorbed water part and, where appropriate, some or all of the organic compound(s) present in the composition.

The x-ray diffractogram of the synthetic phyllosilicate that is suitable for use in the invention has the same positions of the diffraction lines as those of natural talc, with the exception of one line. Specifically, natural talc has a diffraction line at 9.36 Å whereas the synthetic phyllosilicate in accordance with the invention has a diffraction line above 9.4 Å, which may be up to 9.8 Å.

More particularly, the synthetic phyllosilicate in accordance with the invention has a diffraction line greater than 9.4 Å and less than or equal to 9.8 Å.

The synthetic phyllosilicate in accordance with the invention preferably has a diffraction line greater than or equal to 9.5 Å, advantageously greater than or equal to 9.6 Å, and preferentially greater than or equal to 9.7 Å.

The synthetic phyllosilicate in accordance with the invention preferably has a diffraction line less than or equal to 9.7 Å, advantageously less than or equal to 9.6 Å, and preferentially less than or equal to 9.5 Å.

The synthetic phyllosilicate in accordance with the invention may also have a diffraction line between 4.60 Å and 4.80 Å, and/or a diffraction line between 3.10 Å and 3.20 Å and/or a diffraction line between 1.51 Å and 1.53 Å.

It should be noted that a synthetic phyllosilicate in accordance with the invention is free of interfoliar cations. Specifically, this characteristic is demonstrated by the absence of an x-ray diffraction line located at a distance of between 12.00 Å and 18.00 Å, usually revealing a swelling phase with interfoliar spaces in which are found interfoliar cations and possible water molecules.

A synthetic phyllosilicate that is suitable for use in the invention may be present in an amount ranging from 0.01% to 20% by weight, preferably ranging from 0.1% to 15% by weight, more preferentially ranging from 0.1% to 11% by weight, even more preferentially ranging from 0.5% to 11% by weight, better still ranging from 0.5% to 7% by weight, better still ranging from 1% to 6% by weight and even better still ranging from 2% to 5% by weight relative to the total weight of the composition.

It is understood that when a synthetic phyllosilicate in accordance with the invention is in gel form, the “weight %” means the “weight % of solids” or “weight % of active material”.

According to one embodiment, when a synthetic phyllosilicate that is suitable for use in the invention is in aqueous or aqueous-alcoholic gel form, it may constitute only part but also all of the aqueous phase of the composition containing it.

According to a preferred embodiment, when a synthetic phyllosilicate that is suitable for use in the invention is in aqueous or aqueous-alcoholic gel form, it is present in an amount ranging from 0.5 to 20% by weight of active material, preferably from 1% to 15% by weight and even more preferentially ranging from 2% to 10% by weight, relative to the total weight of the aqueous phase.

Polyols and Polyol Derivatives

The term “polyol” is used to refer to organic molecules comprising at least two hydroxyl (OH) functions. The term “polyol” thus includes in particular sugars, and also derivatives thereof.

For the purposes of the invention, the term “polyol” is intended to mean in particular:
a branched or unbranched, saturated or unsaturated, linear hydrocarbon-based chain comprising at least two hydroxyl functions; or
a branched or unbranched, saturated linear hydrocarbon-based chain in which one or more carbon atoms are replaced with an oxygen atom and which comprises at least two hydroxyl functions, for instance polyethylene glycols (PEGs) having from 4 to 8 ethylene glycol units.

Preferably, the polyol of the composition according to the invention has a branched or unbranched, saturated linear hydrocarbon-based chain.

Advantageously, the polyol comprises a number of carbon atoms ranging from 2 to 20, and preferably from 2 to 10, and comprises from 2 to 12, and better still from 2 to 8, hydroxyl functions.

The polyols of the composition according to the invention can be chosen from ethylene glycol, propylene glycol, 1,3-propanediol, isoprene glycol, butylene glycol, dipropylene glycol, polypropylene glycol, glycerol, glycerine, diglycerine, erythritol, pentaerithrytol, arabitol, adonitol, sorbitol, dulcitol, maltitol, panthenol, preferably glycerine, propylene glycol, dipropylene glycol, butylene glycol and 1,3-propanediol, and mixtures thereof.

In particular, the polyol is chosen from propylene glycol, dipropylene glycol and glycerine.

Preferably, the polyol is glycerine.

The polyol derivatives include in particular polyol esters and ethers.

The polyol(s) and derivatives thereof are in particular present in the composition according to the invention in a content of from 0.1% to 30% by weight relative to the total weight of the composition.

Sugars and Sugar Derivatives

For the purposes of the invention, the term “sugar” is used in its commonly accepted most general sense. Thus, the term “sugar” can denote any soluble carbohydrate, generally composed of or derived from simple sugars such as sucrose (saccharose), maltose, glucose and fructose, which includes the polymers of said sugars. Unless otherwise indicated, the sugars under consideration by the invention can be present in their D or L forms.

In this regard, the sugars can be considered to be particular polyols.

The sugars and sugar derivatives can in particular act as a moisturizing active agent or act as an emulsifier, or both at the same time.

According to one particular embodiment of the invention, the sugars and derivatives thereof are chosen from fatty acid esters of sugar and mixtures of fatty acid esters of sugar, which are optionally oxyalkylenated, for example oxyethylenated and/or oxypropylenated, or polyglycerylated, alkyl polyglucosides, and carbohydrates of the family of monosaccharides, or oligosides, or homopolyholosides, which are in particular oxyalkylenated or polyglycerylated, and also mixtures thereof.

According to one particular embodiment, the sugars and sugar derivatives are chosen from alkyl polyglucosides.

According to one particular embodiment, the sugars and sugar derivatives are chosen from carbohydrates of the family of monosaccharides, or oligosides, or homopolyholosides.

Fatty Acid Esters of Sugar

The fatty acid ester(s) of sugar may be monoesters or polyesters of a fatty acid and of a sugar or of an alkyl sugar. They may be oxyalkylenated, for example oxyethylenated and/or oxypropylenated, or polyglycerolated.

They may be chosen in particular from the group comprising esters or mixtures of esters of C₈-C₂₂ fatty acids and of sucrose (saccharose), maltose, glucose or fructose, and esters or mixtures of esters of C₁₄-C₂₂ fatty acids and of (C₁-C₄ alkyl) glucose such as methyl glucose, and mixtures thereof.

In particular, the fatty acid ester of sugar is chosen from sucrose monostearate, sucrose distearate, sucrose tristearate and mixtures thereof, sucrose monolaurate, sucrose monococoate, methyl glucose monostearate, polyglyceryl-3 methyl glucose distearate, methyl o-hexadecanoyl-6-D-glucoside and o-hexadecanoyl-6-D-maltoside, and mixtures thereof, preferably sucrose monostearate.

According to one particular embodiment, the C₈-C₂₂ (preferably C₁₂-C₂₂, and even more preferentially C₁₄-C₂₂) fatty acids forming the fatty unit of the esters that can be used according to the invention comprise a saturated or unsaturated, linear or branched alkyl chain comprising from 8 to 22 carbon atoms (preferably from 12 to 22 carbon atoms, and even more preferentially from 8 to 22 carbon atoms). The fatty unit of the esters may be chosen in particular from stearates, behenates, cocoates, arachidonates, palmitates, myristates, laurates, caprates and oleates, and mixtures thereof. Stearates are preferably used.

According to another particular embodiment, the sugar unit of the fatty acid ester(s) of sugar is chosen from sucrose, maltose, glucose, fructose, mannose, galactose, arabinose, xylose, lactose, trehalose and methyl glucose. Sucrose or glucose is preferably used.

By way of example of fatty acid esters or mixtures of fatty acid esters of sucrose, maltose, glucose or fructose, mention may be made of sucrose monostearate, sucrose distearate, sucrose tristearate and mixtures thereof, such as the products sold in particular by the company Croda under the name Crodesta F50, F70, F110, F160 having respectively an HLB (Hydrophilic Lipophilic Balance) of 5, 7, 11 and 16, the sucrose monostearate sold in particular by the company Evonik Goldschmidt under the reference Tegosoft PSE 141 G, sucrose monolaurate, such as the product sold under the name Grilloten LES 65, and the sucrose monococoate sold in particular under the name Grilloten LES 65K, by the company Grillo-Werke, and by way of example of fatty acid esters or mixtures of fatty acid esters of methyl glucose, mention may be made of methyl glucose monostearate, such as the product sold under the name Grillocose IS by the company Grillo-Werke, or polyglyceryl-3 methyl glucose distearate, such as the product sold in particular by the company Evonik Goldschmidt under the name Tego Care 450.

Mention may also be made of monoesters of glucose or of maltose such as methyl O-hexadecanoyl-6-D-glucoside and O-hexadecanoyl-6-D-maltoside.

Sucrose monostearate is preferred, in particular the product sold by the company Evonik Goldschmidt under the reference Tegosoft PSE 141 G (97% sucrose stearate/3% water).

Alkyl Polyglucosides

The optionally polyalkoxylated alkyl polyglucoside(s) can be chosen from the compounds of general formula below:

R₁O-(G)ₐ

wherein R₁ denotes a linear or branched alkyl and/or alkenyl radical comprising from 4 to 24 carbon atoms, or an alkyl phenyl radical of which the linear or branched alkyl group comprises from 4 to 24 carbon atoms, the G group denotes a sugar comprising from 5 to 6 carbon atoms and a is a number ranging from 1 to 10.

They may be chosen in particular from the group comprising ethers or mixtures of ethers of C₈-C₂₂ fatty alcohol and of glucose, maltose, sucrose or fructose, and ethers or mixtures of ethers of C₁₄-C₂₂ fatty alcohol and of methyl glucose.

The fatty unit of the ethers may be chosen in particular from decyl, cetyl, behenyl, arachidyl, stearyl, palmityl, myristyl, lauryl, capryl, hexadecanoyl and octyldodecyl units, and mixtures thereof such as cetearyl.

The HLB (Hydrophilic Lipophilic Balance) of these surfactants is preferably between 8 and 18.

In particular, the alkyl polyglucoside is chosen from decyl glucoside, lauryl glucoside, cetearyl glucoside, arachidyl glucoside, cocoyl polyglucoside, and mixtures thereof, preferably cetearyl glucoside and arachidyl glucoside.

Examples of alkyl polyglucosides that may be mentioned include decyl glucoside and lauryl glucoside sold, for example, by the company Henkel under the respective names Plantaren 2000 and Plantaren 1200, cetearyl glucoside optionally as a mixture with cetostearyl alcohol, sold, for example, under the name Montanov 68 by the company SEPPIC, under the name Tegocare CG90 by the company Evonik Goldschmidt and under the name Emulgade KE3302 by the company Henkel, and also arachidyl glucoside, for example in the form of the mixture of arachidyl and behenyl alcohols and of arachidyl glucoside, sold in particular under the name Montanov 202 by the company SEPPIC and the mixture of cocoyl polyglucoside and of cetyl and stearyl alcohols (35/65) sold in particular under the name Montanov 82 by the company SEPPIC.

According to one particular embodiment of the invention, the alkyl polyglucoside(s) are chosen from cetearyl glucoside optionally as a mixture with cetostearyl alcohol, sold for example under the name Montanov 68 by the company SEPPIC, under the name Tegocare CG90 by the company Evonik Goldschmidt and under the name Emulgade KE3302 by the company Henkel, and also arachidyl glucoside, for example in the form of the mixture of arachidyl and behenyl alcohols and of arachidyl glucoside, sold in particular under the name Montanov 202 by the company SEPPIC and the mixture of cocoyl polyglucoside and of cetyl and stearyl alcohols (35/65) sold in particular under the name Montanov 82 by the company SEPPIC.

Carbohydrate of the Family of Monosaccharides, or Oligosides, or Homopolyholosides

The term “carbohydrate” is intended to mean any organic molecule containing a carbonyl group (aldehyde or ketone) and several hydroxyl groups (—OH). Carbohydrates were historically known as carbon hydrates. Their chemical formula is based on the model Cₙ(H₂O)ₚ (whence derives the historical name). However, this model is not suitable for all carbohydrates, some of which contain heteroatoms such as nitrogen or phosphorus.

Carbohydrates usually comprise:

(1) monosaccharides, which are simple, non-hydrolyzable crystal-forming molecules. They are of two types: (a) aldoses comprising an aldehyde function on the first carbon and ketoses comprising a ketone function on the second carbon. They are also distinguished according to the number of carbon atoms they contain;

(2) oligosaccharides or oligosides, which are saccharide polymers bearing a sequence of monosaccharides comprising from 2 to 10 monosaccharide units linked via glycoside bonds;

(3) polyholosides (polysaccharides), which are saccharide polymers bearing a sequence of more than 10 monosaccharide units (for example: amylose, amylopectin, cellulose, glycogen).

Among the oligosides and polyosides, the following are distinguished:
homopolyosides are carbohydrates whose hydrolysis gives only one type of saccharide;
heterosides and heteropolyosides are carbohydrates whose hydrolysis does not give only one type of saccharide. These are polymers of monosaccharides and of non-saccharide molecules. An example of a heteroside that may be mentioned is salicin.

The invention relates to the carbohydrates of the family of:
monosaccharides,
oligosaccharides or oligosides,
polysaccharides of homopolyholoside type.

(1) Monosaccharides

Among the monosaccharides that may be used according to the invention, mention may be made of:
trioses containing 3 carbons: dihydroxyacetone, glyceraldehyde;
tetroses containing 4 carbons: erythrose, threose, erythrulose;
pentoses containing 5 carbons: ribose, arabinose, xylose, lyxose, ribulose, xylulose, deoxyribose;
hexoses containing 6 carbons: allose, altrose, glucose, mannose, fucose, gulose, idose, galactose, talose, fuculose, psicose, fructose, sorbose, tagatose, quinovose, pneumose, rhamnose;
heptoses containing 7 carbons: sedoheptulose, glucoheptose, idoheptulose, mannoheptulose, taloheptulose;
octoses containing 8 carbons;
monosaccharides having more than 8 carbons, for instance maltitol;

in their D or L form.

Among these monosaccharides, use will preferentially be made of trehalose and/or hexoses, and more particularly glucose, mannose, rhamnose and fructose.

Mention may also be made of derivatives thereof, in particular alkylated derivatives thereof, such as methylated derivatives, for instance methyl glucose, and also compounds containing one or more sugars, and mixtures thereof. As compound containing a sugar or a mixture of sugars, mention may be made of natural compounds such as honey, and polymers, for instance the product sold under the name Fucogel 1000 by the company Solabia (CTFA name Biosaccharide gum-1), which is a polymer containing fucose, galactose and galacturonic acid.

(2) Oligosaccharides

Among the oligosaccharides that may be used according to the invention, mention may be made of:

(i) disaccharides or diholosides or diosides composed of two monosaccharide molecules and which may be reducing or non-reducing. The term “non-reducing disaccharide” is intended to mean any disaccharide of which the carbon 1 bearing the hemiacetal OH is involved in a bond, namely the hemiacetal function is therefore not free. The term “reducing disaccharide” is intended to mean any disaccharide of which the hemiacetal function is free.

Among the non-reducing disaccharides, mention may be made of sucrose and trehalose. Among the reducing disaccharides, mention may be made of lactose, maltose, cellobiose, isomaltose and melibiose.

(ii) triholosides composed of three monosaccharide molecules, such as raffinose, gentianose or melezitose.

(iii) dextrins and cyclodextrins which are mixtures of linear gluco-oligosides (glucose oligosides) of which the glucose units are bonded by oside bonds of the α-(1,4) type, but of which the group is bonded by an α-(1,6) oside bond.

(3) Homopolysaccharides or Homoglycans

Among the polysaccharides or polyholosides, consideration is given herein to homopolysaccharides (or homoglycans) constituted of the same monosaccharide: fructans, glucans, galactans, mannans, for example, which may be linear, branched or mixed.

Mention may be made, for example, of:
homopolymers of fructan, including inulin, for example, which is a polyholoside composed of fructose units connected by a *β(2→1) linker, the fructose chain ending with an α-D-glucose;
homopolymers of glucan, including starches, for example, which are non-reducing homogeneous polyholosides constituted of two compounds: amylose (water-soluble), which is a polymer of glucose linked by an α(1→4) bond (20% to 30%), and amylopectin (insoluble), which is amylose branched via an α(1→6) bond (70% to 80%). Mention may also be made of glycogen, which in structural terms is virtually identical to starch: it has more branches than starch (one branch every 10 glucose residues), all the rest of the structure being identical to starch. Its molar mass is higher (about 106 g·mol⁻¹). Cellulose, likewise, which is a homogeneous polyholoside of glucose linked by a β(1→4) bond. Or, alternatively, dextrans, which are compounds of D-glucose units connected by an α(1→6) oside bond;
homopolymers of galactan, including agar-agar, for example, which is a mixed polyholoside constituted of D- and L-galactose esterified with sulfuric acid, or carrageenans;
or else homopolymers of xylose (xylans), or of mannose (mannans).

According to one particular embodiment of the invention, the carbohydrate(s) are chosen from monosaccharides.

According to one particular embodiment of the invention, the compound(s) chosen from sugars and derivatives thereof are chosen from sucrose esters, glucose esters, glucose ethers, rhamnose, mannose, trehalose and fucose.

The sugar-derived emulsifiers are chosen from:
esters of sucrose or of glucose and of fatty acid (for example, sucrose palmitostearate);
alkyl polyglucosides (APGs), such as the Montanov(s) from SEPPIC, Montanov 202, 68 or else 82.

In a non-exhaustive manner, the following alkyl polyglucosides may be suitable as emulsifiers for the purposes of the invention.

| Trade name | Active agent | Supplier |
|---|---|---|
| MONTANOV 68 | CETEARYL ALCOHOL (and) CETEARYL GLUCOSIDE | SEPPIC |
| MONTANOV 68 | MIXTURE OF CETYLSTEARYL GLUCOSIDE AND OF | SEPPIC |
| | CETYL AND STEARYL ALCOHOLS (12/46/42) | |
| MONTANOV 14 | MIXTURE OF MYRISTYL POLYGLUCOSIDE AND OF | SEPPIC |
| | MYRISTYL ALCOHOL (30/70) | |
| MONTANOV 202 | MIXTURE OF ARACHYDYL POLYGLUCOSIDE AND | SEPPIC |
| | OF ARACHIDYL AND BEHENYL ALCOHOLS (15/85) | |
| MONTANOV 68 | MIXTURE OF CETYLSTEARYL GLUCOSIDE AND OF | SEPPIC |
| | CETYL AND STEARYL ALCOHOLS (12/46/42) | |
| MONTANOV 202 | MIXTURE OF ARACHYDYL POLYGLUCOSIDE AND | SEPPIC |
| | OF ARACHIDYL AND BEHENYL ALCOHOLS (15/85) | |
| MONTANOV 82 | MIXTURE OF COCOYL POLYGLUCOSIDE AND OF | SEPPIC |
| | CETYL AND STEARYL ALCOHOL (35/65) | |
| MONTANOV 14 | MIXTURE OF MYRISTYL POLYGLUCOSIDE AND OF | SEPPIC |
| | MYRISTYL ALCOHOL (30/70) | |
| MONTANOV L | MIXTURE OF ALKYL GLUCOSIDES (C12 TO C20)/ | SEPPIC |
| | FATTY ALCOHOL (C14 TO C22) | |
| ORAMIX CG 110 | (C8/C10 50/50) ALKYL POLYGLUCOSIDE (2) AS A | SEPPIC |
| | BUFFERED 60% AQUEOUS SOLUTION | |
| ORAMIX NS 10 | (C10 /12/14 85/10/5) ALKYL POLYGLUCOSIDE (1.4) AS | SEPPIC |
| | A 50% AQUEOUS SOLUTION, NON-STABILIZED | |
| PLANTACARE | (C8/C10/C12/C14 34/24/29/10) ALKYL | COGNIS (BASF) |
| 2000 UP | POLYGLUCOSIDE (1.4) AS A 53% AQUEOUS | |
| | SOLUTION, NON-PROTECTED (PH 11.5 WITH NAOH) | |
| PLANTACARE 818 | (C8/C16) ALKYL POLYGLUCOSIDE (1.4) AS A 53% | COGNIS (BASF) |
| UP | AQUEOUS SOLUTION, NON-PROTECTED (PH 11.5 | |
| | TO 12.5) | |
| MYDOL 10 | (C9/C11) ALKYL POLYGLUCOSIDE (1.4) AS A 40% | KAO |
| | AQUEOUS SOLUTION (PH 10) | |
| TEGO CARE CG 90 | MIXTURE OF CETYLSTEARYL GLUCOSIDE/ | EVONIK |
| | CETYLSTEARYL ALCOHOL | GOLDSCHMIDT |

A composition for the purposes of the invention may comprise at least one moisturizing agent of sugar type.

For the purposes of the present invention, the term “moisturizing agent of sugar type” is intended to mean hygroscopic substances of the family of:
simple sugars,
sugar polyols,
glycosaminoglycans (GAGs), or mucopolysaccharides which have a high water retention capacity. Among these, mention may be made of sulfated GAGs and hyaluronic acid constituting the skin, and its salts.

Preferably, a polyol suitable for use in the invention is a sugar or a sugar derivative, in particular chosen from monosaccharides, disaccharides, oligosaccharides, polysaccharides and glycosaminoglycans; said oligosaccharides and polysaccharides possibly being linear, branched or mixed, and constituted of the same monosaccharide or of different monosaccharides, in particular of the same monosaccharide, and also mixtures thereof.

Advantageously, a sugar suitable for use in the invention is a fatty acid ester of sugar chosen from: esters or mixtures of esters of C₈-C₂₂ fatty acid and of sucrose, maltose, glucose or fructose, and esters or mixtures of esters of C₁₄-C₂₂ fatty acid and of (C₁-C₄ alkyl) glucose, and mixtures thereof.

According to one particular embodiment, the polyol(s) suitable for use in the invention are monosaccharides chosen from: trioses, tetroses, pentoses, hexoses, heptoses and octoses, and monosaccharides having more than 8 carbons, said monosaccharides possibly being present in their D or L forms.

Thus, the sugars are in particular chosen from Rhamnose, Mannose, Trehalose, Talose, Fucose, Ribose, Idose, Arabinose, Gulose, Xylose, Lyxose, Altrose, Allose, Glucose, Mannose, Galactose, Lactose, Sucrose, Cellobiose, Maltose, Fucose α(1-3) Glucose and Fructose, even more preferentially Rhamnose and Mannose, and also mixtures thereof.

The content of sugar, or sugar derivative, in the composition as described in the invention, can in particular range from 0.1% to 30%, relative to the total weight of the composition.

UV-Screening Agents

The compositions according to the invention contain at least one UV-screening agent. More particularly, the UV-screening agent suitable for use in the invention is chosen from water-soluble organic UV-screening agents, liposoluble organic UV-screening agents, insoluble organic UV-screening agents, inorganic UV-screening agents, and mixtures thereof. Preferably, the UV-screening agent suitable for use in the invention is chosen from water-soluble organic UV-screening agents, liposoluble organic UV-screening agents, insoluble organic UV-screening agents, and mixtures thereof.

Even more preferentially, the UV-screening agent suitable for use in the invention is chosen from water-soluble organic UV-screening agents, liposoluble organic UV-screening agents, and mixtures thereof.

The term “water-soluble organic UV-screening agent” is intended to mean any organic compound for screening out UV radiation which can be fully dissolved in molecular form or miscible in a liquid aqueous phase or else can be dissolved in colloidal form (for example in micellar form) in a liquid aqueous phase.

The term “liposoluble organic UV-screening agent” is intended to mean any cosmetic or dermatological, organic or inorganic compound for screening out UV radiation which can be fully dissolved in molecular form or miscible in an oily phase or else can be dissolved in colloidal form (for example in micellar form) in an oily phase.

The term “insoluble organic UV-screening agent” is intended to mean any cosmetic or dermatological, organic or inorganic compound for screening out UV radiation which has a solubility in water of less than 0.5% by weight and a solubility of less than 0.5% by weight in the majority of organic solvents such as liquid paraffin, fatty alcohol benzoates and fatty acid triglycerides, for example Miglyol 812® sold by the company Dynamit Nobel. This solubility, determined at 70° C., is defined as the amount of product in solution in the solvent at equilibrium with an excess of solid in suspension after returning to ambient temperature. It may be readily evaluated in the laboratory.

### I/ Water-Soluble Organic UV-Screening Agents

### A/ Water-Soluble Organic UVA-Screening Agents

The term “water-soluble organic UVA-screening agent” is intended to mean any organic compound for screening out UVA radiation in the wavelength range 320 to 400 nm which can be fully dissolved in molecular form or miscible in a liquid aqueous phase or else can be dissolved in colloidal form (for example in micellar form) in a liquid aqueous phase.

Among the water-soluble organic UVA-screening agents that may be used according to the present invention, mention may be made of:

benzene-1,4-bis(3-methylidene-10-camphorsulfonic acid) (INCI name: Terephthalylidene Dicamphor Sulfonic Acid) and the various salts thereof, described in particular in patent applications FR-A-2528420 and FR-A-2639347.

These screening agents correspond to general formula (I) below:

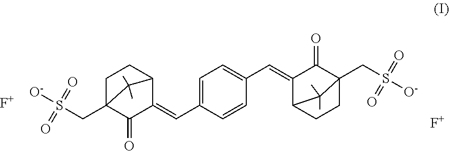

wherein F denotes a hydrogen atom, an alkali metal or else a radical NH(R₁)₃⁺ in which the radicals R₁, which may be identical or different, denote a hydrogen atom, a C₁-C₄ alkyl or hydroxyalkyl radical or else a group Mⁿ⁺/n, Mⁿ⁺ denoting a polyvalent metal cation in which n is equal to 2 or 3 or 4, Mⁿ⁺ preferably denoting a metal cation chosen from Ca²⁺, Zn²⁺, Mg²⁺, Ba²⁺, Al³⁺ and Zr⁴⁺. It is clearly understood that the compounds of formula (I) above can give rise to the “cis-trans” isomer around one or more double bond(s) and that all the isomers are within the context of the present invention.

Among the hydrophilic organic UVA-screening agents that can be used according to the present invention, mention may also be made of compounds comprising at least two benzazolyl groups bearing sulfonic groups, such as those described in patent application EP-A-0 669 323. They are described and prepared according to the syntheses indicated in U.S. Pat. No. 2,463,264 and also in patent application EP-A-0 669 323.

The compounds comprising at least two benzazolyl groups in accordance with the invention correspond to general formula (II) below:

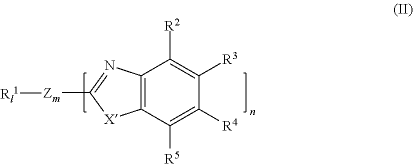

in which:
Z represents an organic residue of valency (1+n) comprising one or more double bonds placed such that it completes the system of double bonds of at least two benzazolyl groups as defined inside the square brackets so as to form a totally conjugated assembly;
X′ denotes S, O or NR6;
R¹ denotes hydrogen, C₁-C₁₈ alkyl, C₁-C₄ alkoxy, a C₅-C₁₅ aryl, a C₂-C₁₈ acyloxy, SO₃Y or COOY;
the radicals R², R³, R⁴ and R⁵, which may be identical or different, denote a nitro group or a radical R¹;
R⁶ denotes hydrogen, a C₁-C₄ alkyl or a C₁-C₄ hydroxyalkyl;
Y denotes hydrogen, Li, Na, K, NH₄, ½Ca, ½Mg, ⅓Al or a cation resulting from the neutralization of a free acid group with an organic nitrogenous base;
m is 0 or 1;
n is a number from 2 to 6;
l is a number from 1 to 4;
with the proviso that 1+n does not exceed the value 6.

Among these compounds, preference is given to those for which the group Z is chosen from the group made up of:

(a) an olefin linear aliphatic C₂-C₆ hydrocarbon-based radical which may be interrupted with a C₅-C₁₂ aryl group or a C₄-C₁₀ heteroaryl, in particular chosen from the following groups:

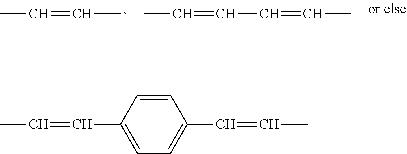

(b) a C₅-C₁₅ aryl group which may be interrupted with an olefin linear aliphatic C₂-C₆ hydrocarbon-based radical, in particular chosen from the following groups:

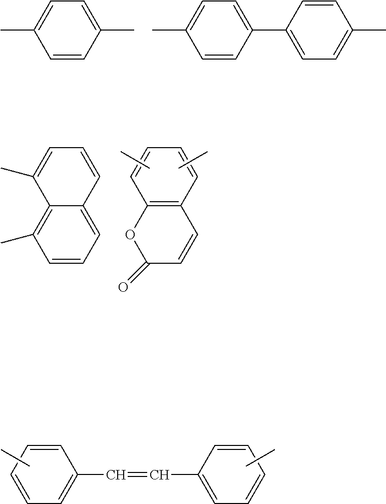

(c) a C₃-C₁₀ heteroaryl residue, in particular chosen from the following groups:

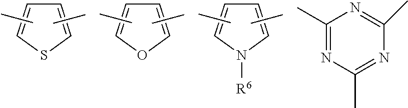

in which R⁶ has the same meaning as that indicated above; said radicals Z as defined in paragraphs (a), (b) and (c) possibly being substituted with C₁-C₆ alkyl, C₁-C₆ alkoxy, phenoxy, hydroxyl, methylenedioxy or amino radicals optionally substituted with one or two C₁-C₅ alkyl radicals.

Preferably, the compounds of formula (II) comprise, per molecule, 1, 3 or 4 SO₃Y groups.

As examples of compounds of formula (II) that may be used, mention may be made of the compounds of formulae (a) to (j) having the following structure, and also the salts thereof:

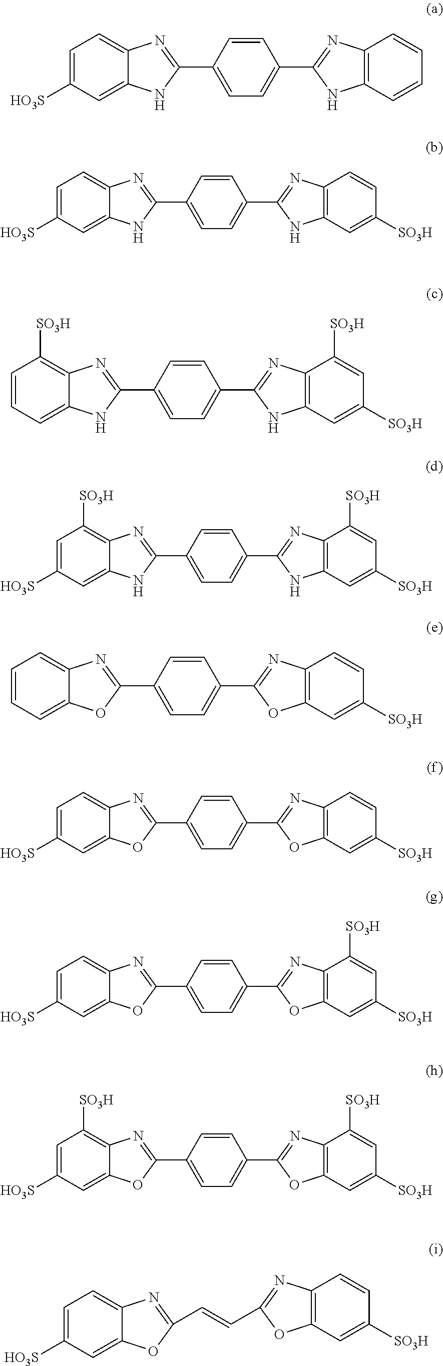

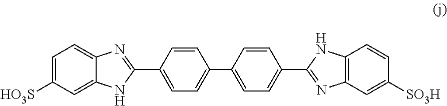 [SMILES: O=S(=O)(O)c1ccc2[nH]c(-c3ccc(-c4ccc(-c5nc6ccc(S(=O)(=O)O)cc6[nH]5)cc4)cc3)nc2c1]

Among all these compounds, preference will most particularly be given to 1,4-bis-benzimidazolyl-phenylene-3,3′,5,5′-tetrasulfonic acid (INCI name: Disodium Phenyl Dibenzimidazole Tetrasulfonate) (compound (d)) or a salt thereof, having the following structure, sold in particular under the name Neoheliopan AP® by the company Symrise:

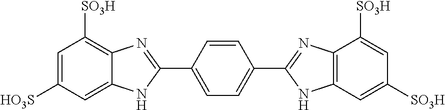 [SMILES: O=S(=O)(O)c1cc(S(=O)(=O)O)c2nc(-c3ccc(-c4nc5c(S(=O)(=O)O)cc(S(=O)(=O)O)cc5[nH]4)cc3)[nH]c2c1]

Among the water-soluble organic UVA-screening agents that may be used according to the present invention, mention may also be made of benzophenone compounds comprising at least one sulfonic acid function, for instance the following compounds:

Benzophenone-4, sold in particular by the company BASF under the name Uvinul MS40®:

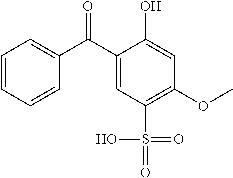 [SMILES: COc1cc(O)c(C(=O)c2ccccc2)cc1S(=O)(=O)O]

Benzophenone-5 having the structure

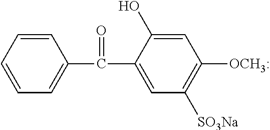

Benzophenone-9, sold in particular by the company BASF under the name Uvinul DS49:

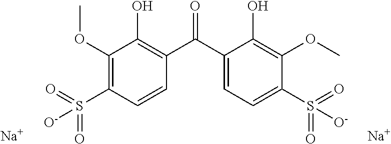 [SMILES: [Na+] ; COc1c(S(=O)(=O)[O-])ccc(C(=O)c2ccc(S(=O)(=O)[O-])c(OC)c2O)c1O ; [Na+]]

Among the water-soluble organic UVA-screening agents, use will more particularly be made of benzene-1,4-bis(3-methylidene-10-camphorsulfonic acid) and the various salts thereof (INCI name: Terephthalylidene Dicamphor Sulfonic Acid) produced by the company Chimex under the trade name Mexoryl SX®.

### B/ Water-Soluble Organic UVB-Screening Agents

The water-soluble organic UVB-screening agents are in particular chosen from: water-soluble cinnamic derivatives such as ferulic acid or 3-methoxy-4-hydroxycinnamic acid,

water-soluble benzylidenecamphor compounds;
water-soluble phenylbenzimidazole compounds;
water-soluble p-aminobenzoic (PABA) compounds;
water-soluble salicylic compounds and mixtures thereof.

As examples of water-soluble organic UVB-screening agents, mention may be made of those denoted hereinbelow under their INCI name:

### Para-Aminobenzoic Compounds:

PABA,
PEG-25 PABA, sold in particular under the name Uvinul P 25® by BASF.

### Salicylic Compounds:

Dipropylene glycol salicylate, sold in particular under the name Dipsal® by Scher,
TEA salicylate, sold in particular under the name Neo Heliopan TS® by Symrise,

### Benzylidenecamphor Compounds:

Benzylidenecamphorsulfonic acid, sold in particular under the name Mexoryl SL® by Chimex,
Camphor benzalkonium methosulfate, sold in particular under the name Mexoryl SO® by Chimex.

### Phenylbenzimidazole Compounds:

Phenylbenzimidazolesulfonic acid, sold in particular under the trade name Eusolex 232® by Merck.

Use will more particularly be made of the screening agent phenylbenzimidazolesulfonic acid, sold in particular under the trade name Eusolex 232® by Merck.

### II/ Liposoluble Organic UV-Screening Agents

The liposoluble organic UV-screening agents are in particular chosen from cinnamic derivatives; anthranilates; salicylic derivatives; dibenzoylmethane derivatives; camphor derivatives; benzophenone derivatives; β, β-diphenylacrylate derivatives; triazine derivatives; benzotriazole derivatives; benzalmalonate derivatives, in particular those mentioned in U.S. Pat. No. 5,624,663; imidazolines; p-aminobenzoic acid (PABA) derivatives; benzoxazole derivatives as described in patent applications EP0832642, EP1027883, EP1300137 and DE10162844; screening polymers and screening silicones such as those described in particular in application WO-93/04665; α-alkylstyrene-based dimers such as those described in patent application DE19855649; 4,4-diarylbutadienes such as those described in applications EP0967200, DE19746654, DE19755649, EP-A-1008586, EP1133980 and EP133981; merocyanine derivatives, merocyanines as described in U.S. Pat. No. 4,195,999, application WO2004/006878, applications WO2008/090066, WO2011113718 and WO2009027258, and the documents IP COM JOURNAL No 000179675D published on Feb. 23, 2009, IP COM JOURNAL No 000182396D published on Apr. 29, 2009, IP COM JOURNAL No 000189542D published on Nov. 12, 2009, IP COM Journal No IPCOM000011179D published on Mar. 4, 2004; and mixtures thereof.

As examples of additional organic photoprotective agents, mention may be made of those denoted hereinbelow under their INCI name:

### Dibenzoylmethane Derivative:

Butylmethoxydibenzoylmethane or avobenzone sold in particular under the trade name Parsol 1789 by the company DSM Nutritional Products;

### Para-Aminobenzoic Acid Derivatives:

Ethyl PABA,
Ethyl dihydroxypropyl PABA,
Ethylhexyl dimethyl PABA sold in particular under the name Escalol 507 by ISP;

### Salicylic Derivatives:

Homosalate sold in particular under the name Eusolex HMS by Rona/EM Industries,
Ethylhexyl salicylate sold in particular under the name Neo Heliopan OS by Symrise;

### Cinnamic Derivatives:

Ethylhexyl methoxycinnamate, sold in particular under the trade name Parsol MCX by DSM Nutritional Products,
Isopropyl methoxycinnamate,
Isoamyl methoxycinnamate sold in particular under the trade name Neo Heliopan E 1000 by Symrise,
Cinoxate,
Diisopropyl methyl cinnamate;

### β,β-Diphenylacrylate Derivatives:

Octocrylene sold in particular under the trade name Uvinul N539 by BASF,
Etocrylene sold in particular under the trade name Uvinul N35 by BASF;

### Benzophenone Derivatives:

Benzophenone-1 sold in particular under the trade name Uvinul 400 by BASF,
Benzophenone-2 sold in particular under the trade name Uvinul D50 by BASF,
Benzophenone-3 or oxybenzone sold in particular under the trade name Uvinul M40 by BASF,
Benzophenone-6 sold in particular under the trade name Helisorb 11 by Norquay,
Benzophenone-8 sold in particular under the trade name Spectra-Sorb UV-24 by American Cyanamid,
Benzophenone-12,
n-hexyl 2-(4-diethylamino-2-hydroxybenzoyl)benzoate sold in particular under the trade name Uvinul A+, such as Uvinul A+Granular, or in the form of a mixture with octyl methoxycinnamate in particular under the trade name Uvinul A+B by BASF;

### Benzylidene Camphor Derivatives:

3-Benzylidenecamphor sold in particular under the name Mexoryl SD by Chimex,
4-Methylbenzylidenecamphor sold in particular under the name Eusolex 6300 by Merck,
   Polyacrylamidomethylbenzylidenecamphor sold in particular under the name Mexoryl SW by Chimex;

### Phenylbenzotriazole Derivatives:

Drometrizole trisiloxane, sold in particular under the name Silatrizole by Rhodia Chimie;

### Triazine Derivatives:

Bis(ethylhexyloxyphenol)methoxyphenyltriazine sold in particular under the trade name Tinosorb S by BASF,
Ethylhexyl triazone sold in particular under the trade name Uvinul T150 by BASF,
Diethylhexyl butamido triazone sold in particular under the trade name Uvasorb HEB by Sigma 3V,
   Silicone triazines substituted with two aminobenzoate groups, as described in patent EP0841341, in particular 2,4-bis(n-butyl 4′-aminobenzalmalonate)-6-[(3-{1,3,3,3-tetramethyl-1-[(trimethyl silyl)oxy]disiloxanyl}propyl)amino]-s-triazine;

### Anthranilic Derivatives:

Menthyl anthranilate sold in particular under the trade name Neo Heliopan MA by Symrise;

### Imidazoline Derivatives:

Ethylhexyl dimethoxybenzylidene dioxoimidazoline propionate;

### Benzalmalonate Derivatives:

Dineopentyl 4′-methoxybenzalmalonate,
Polyorganosiloxane containing benzalmalonate functions, for instance Polysilicone-15 sold in particular under the trade name Parsol SLX by DSM;

### 4,4-Diarylbutadiene Derivatives:

1,1-Dicarboxy(2,2′-dimethylpropyl)-4,4-diphenylbutadiene;

### Benzoxazole Derivatives:

2,4-Bis[5-(1-dimethylpropyl)benzoxazol-2-yl-(4-phenyl)imino]-6-(2-ethylhexyl)imino-1,3,5-triazine sold in particular under the name Uvasorb K2A by Sigma 3V,
and mixtures thereof;

### Lipophilic Merocyanine Derivatives:

Octyl 5-N,N-diethylamino-2-phenyl sulfonyl-2,4-pentadienoate;
and mixtures thereof;

The preferential liposoluble organic screening agents are chosen from:
Butyl methoxydibenzoylmethane
Ethylhexyl methoxycinnamate
Ethylhexyl salicylate,
Homosalate,
Butyl methoxydibenzoylmethane
octocrylene,
Benzophenone-3,
n-Hexyl 2-(4-diethylamino-2-hydroxybenzoyl)benzoate,
4-Methylbenzylidene camphor,
bis-Ethylhexyloxyphenol methoxyphenyl triazine,
Ethylhexyl triazone,
Diethylhexyl butamido triazone,
2,4,6-tris(dineopentyl 4′-aminobenzalmalonate)-s-triazine,
2,4,6-tris(diisobutyl 4′-aminobenzalmalonate)-s-triazine,
2,4-bis(dineopentyl 4′-aminobenzalmalonate)-6-(n-butyl 4′-aminobenzoate)-s-triazine,
Drometrizole trisiloxane,
Polysilicone-15,
1,1-Dicarboxy(2,2′-dimethylpropyl)-4,4-diphenylbutadiene,
2,4-bis[5-(1-Dimethylpropyl)benzoxazol-2-yl-(4-phenyl)imino]-6-(2-ethylhexyl)imino-1,3,5-triazine,
and mixtures thereof.

The preferred lipophilic organic screening agents are more particularly chosen from:
Butyl methoxydibenzoylmethane
octocrylene,
Ethylhexyl salicylate,
n-Hexyl 2-(4-diethylamino-2-hydroxybenzoyl)benzoate,
bis-Ethylhexyloxyphenol methoxyphenyl triazine,
Ethylhexyl triazone,
Diethylhexyl butamido triazone,
Drometrizole trisiloxane, and mixtures thereof.

Among these liposoluble organic UV-screening agents, some of them are liquid at ambient temperature (20-25° C.) under 1 atmosphere.

### III/ Insoluble Organic UV-Screening Agents

The insoluble organic UV-screening agents according to the invention preferably have a mean particle size which ranges from 0.01 to 5 μm and more preferentially from 0.01 to 2 μm and more particularly from 0.020 to 2 μm.

The mean particle diameter is measured using a particle size distribution analyzer of the Culter N4 PLUS type manufactured by Beckman Coulter Inc.

The insoluble organic screening agents according to the invention can be brought to the desired particulate form by any ad hoc means, in particular such as dry milling or milling in a solvent medium, sieving, atomization, micronization or spraying.

The insoluble organic screening agents according to the invention in micronized form may in particular be obtained by means of a process of milling an insoluble organic UV-screening agent in the form of particles of coarse size in the presence of an appropriate surfactant making it possible to improve the dispersion of the resulting particles in the cosmetic formulations.

An example of a process for micronization of insoluble organic screening agents is described in applications GB-A-2 303 549 and EP-A-893119. The milling apparatus used according to these documents may be a jet, ball, vibration or hammer mill and preferably a high speed stirring mill or an impact mill and more particularly a rotating ball mill, a vibrating mill, a tube mill or a rod mill.

According to this particular process, use is made, as surfactants for milling said screening agents, of alkyl polyglucosides having the structure CₙH₂ₙ₊₁ O(C₆H₁₀O₅)ₓH in which n is an integer from 8 to 16 and x is the mean degree of polymerization of the unit (C₆H₁₀O₅) and ranges from 1.4 to 1.6. They may be chosen from C₁-C₁₂ esters of a compound having the structure CₙH₂ₙ₊₁ O(C₆H₁₀O₅)ₓH and more particularly an ester obtained by reacting a C₁-C₁₂ carboxylic acid, such as formic, acetic, propionic, butyric, sulfosuccinic, citric or tartaric acid, with one or more free OH functions on the glucoside unit (C₆H₁₀O₅). Decylglucoside may in particular be mentioned as alkyl polyglucoside.

Said surfactants are generally used at a concentration ranging from 1% to 50% by weight and more preferentially from 5% to 40% by weight, relative to the insoluble screening agent in its micronized form.

The insoluble organic UV-screening agents in accordance with the invention may be chosen in particular from organic UV-screening agents of the oxalanilide type, of the triazine type, of the benzotriazole type; of the vinylamide type; of the cinnamide type; of the type comprising one or more groups which are benzazole and/or benzofuran, benzothiophene or of the indole type; of the aryl vinylene ketone type; of the phenylene bis-benzoxazinone derivative type; of the amide, sulfonamide or acrylonitrile carbamate derivative type, or mixtures thereof.

For the purpose for which it is used in the present invention, the term “benzazole” encompasses at the same time benzothiazoles, benzoxazoles and benzimidazoles.

### A/ Oxalanides

Among the UV-screening agents of the oxalanilide type in accordance with the invention, mention may be made of those corresponding to the structure:

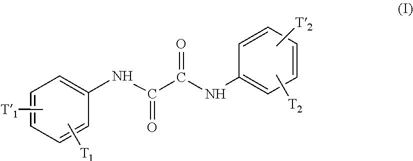

in which T₁, T′₁, T₂ and T′₂ denote, identically or differently, a C₁ to C₈ alkyl radical or a C₁ to C₈ alkoxy radical. These compounds are described in patent application WO 95/22959.

By way of examples, mention may be made of the commercial products Tinuvin 315® and Tinuvin 312® sold by the company BASF and respectively having the structure:

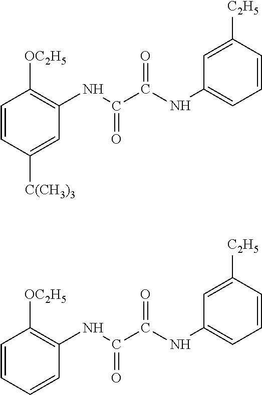 [SMILES: CCOc1ccc(C(C)(C)C)cc1NC(=O)C(=O)Nc1cccc(CC)c1 ; CCOc1ccccc1NC(=O)C(=O)Nc1cccc(CC)c1]

### B/ Triazines

Among the insoluble UV-screening agents of the triazine type in accordance with the invention, mention may also be made of those corresponding to formula (II) below:

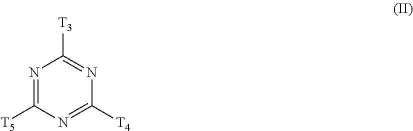

wherein T₃, T₄ and T₅, independently, are phenyl, phenoxy or pyrrolo, in which the phenyl, phenoxy and pyrrolo are unsubstituted or substituted with one, two or three substituents chosen from OH, C₁-C₁₈ alkyl or C₁-C₁₈ alkoxy, C₁-C₁₈ carboxyalkyl, C₅-C₈ cycloalkyl, a methylbenzylidenecamphor group, or a —(CH═CH)ₙ(CO)—OT₆ group, with T₆ either C₁-C₁₈ alkyl or cinnamyl.

These compounds are described in WO 97/03642, GB 2286774, EP-743309, WO 98/22447 and GB 2319523.

Among the UV-screening agents of the triazine type in accordance with the invention, mention may also be made of insoluble derivatives of s-triazine bearing benzalmalonate and/or phenyl cyanoacrylate groups, such as those described in application EP-A-0790243 (which is an integral part of the content of the description).

Among these insoluble UV-screening agents of the triazine type, mention will more particularly be made of the following compounds:
2,4,6-tris(diethyl 4′-aminobenzalmalonate)-s-triazine,
2,4,6-tris(diisopropyl 4′-aminobenzalmalonate)-s-triazine,
2,4,6-tris(dimethyl 4′-aminobenzalmalonate)-s-triazine,
2,4,6-tris(ethyl α-cyano-4-aminocinnamate)-s-triazine.

Among the UV-screening agents of the triazine type in accordance with the invention, mention may also be made of insoluble derivatives of s-triazine bearing benzotriazole and/or benzothiazole groups, such as those described in application WO 98/25922 (which forms an integral part of the content of the description).

Among these compounds, mention may more particularly be made of:
2,4,6-tris[(3′-benzotriazol-2-yl-2′-hydroxy-5′-methyl)phenylamino]-s-triazine,
2,4,6-tris[(3′-benzotriazol-2-yl-2′-hydroxy-5′-tert-octyl)phenylamino]-s-triazine.

Mention may also be made of the symmetrical triazines substituted with naphthalenyl groups or polyphenyl groups described in U.S. Pat. No. 6,225,467, application WO2004/085412 (see compounds 6 and 9) or the document “Symmetrical Triazine Derivatives” IP.COM Journal, IP.COM INC West Henrietta, N.Y., US (Sep. 20, 2004), in particular 2,4,6-tris(di-phenyl)triazine and 2,4,6-tris(ter-phenyl)triazine which is reiterated in patent applications WO06/035000, WO06/034982, WO06/034991, WO06/035007, WO2006/034992, and WO2006/034985.

### C/ Benzotriazoles

Among the insoluble organic UV-screening agents of the benzotriazole type in accordance with the invention, mention may be made of those of formula (III) below, as described in application WO 95/22959 (which forms an integral part of the content of the description):

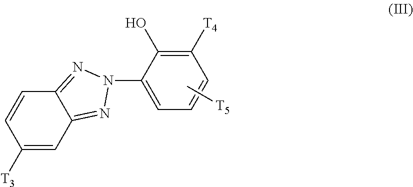

wherein T₇ denotes a hydrogen atom or a C₁ to C₁₈ alkyl radical; and T₈ and T₉, which may be identical or different, denote a C₁ to C₁₈ alkyl radical optionally substituted with a phenyl.
As examples of compounds of formula (III), mention may be made of the commercial products Tinuvin 328, 320, 234 and 350 from the company BASF, having the structure below:

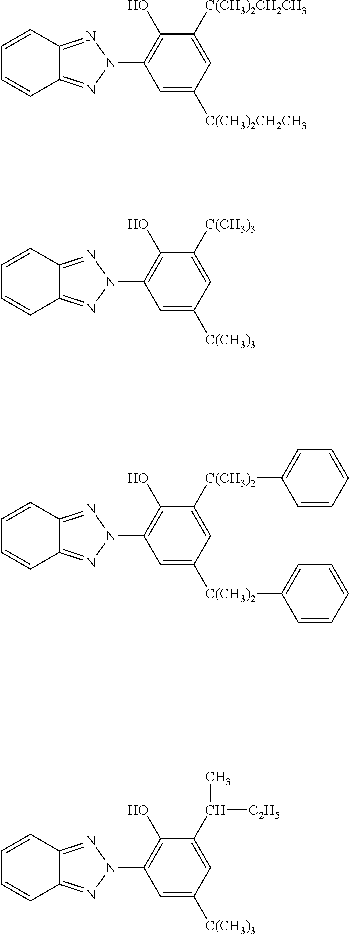

Among the insoluble organic UV-screening agents of the benzotriazole type in accordance with the invention, mention may be made of the compounds as described in patents U.S. Pat. No. 5,687,521, U.S. Pat. No. 5,373,037 and U.S. Pat. No. 5,362,881, and in particular [2,4′-dihydroxy-3-(2H-benzotriazol-2-yl)-5-(1,1,3,3-tetramethylbutyl)-2′-n-octoxy-5′-benzoyl]diphenylmethane sold in particular under the name Mixxim PB30® by the company Fairmount Chemical, having the structure:

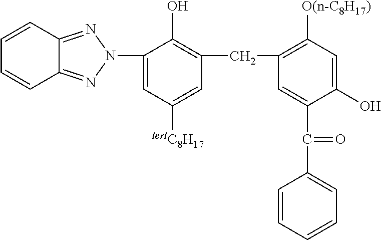

Among the insoluble organic UV-screening agents of the benzotriazole type in accordance with the invention, mention may be made of the methylenebis(hydroxyphenylbenzotriazole) derivatives having the structure below:

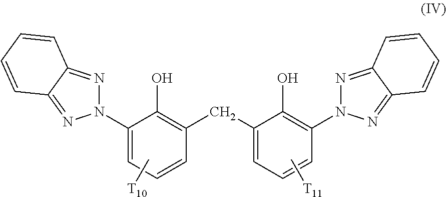

wherein the radicals T₁₀ and T₁₁, which may be identical or different, denote a C₁ to C₁₈ alkyl radical which may be substituted with one or more radicals chosen from C₁-C₄ alkyl, C₅-C₁₂ cycloalkyl or an aryl residue. These compounds are known per se and described in applications 5 U.S. Pat. No. 5,237,071, U.S. Pat. No. 5,166,355, GB-A-2 303 549, DE 197 26 184 and EP-A-893 119 (which are an integral part of the description).

In formula (I) defined above: the C₁-C₁₈ alkyl groups may be linear or branched and are, for example, methyl, ethyl, n-propyl, isopropyl, n-butyl, isobutyl, tert-butyl, tert-octyl, n-amyl, n-hexyl, n-heptyl, n-octyl, isooctyl, n-nonyl, n-decyl, n-undecyl, n-dodecyl, tetradecyl, hexadecyl or octadecyl; the C₅-C₁₂ cycloalkyl groups are, for example, cyclopentyl, cyclohexyl or cyclooctyl; the aryl groups are, for example, phenyl or benzyl.

Among the compounds of formula (IV), mention may be made of those having the structure below:

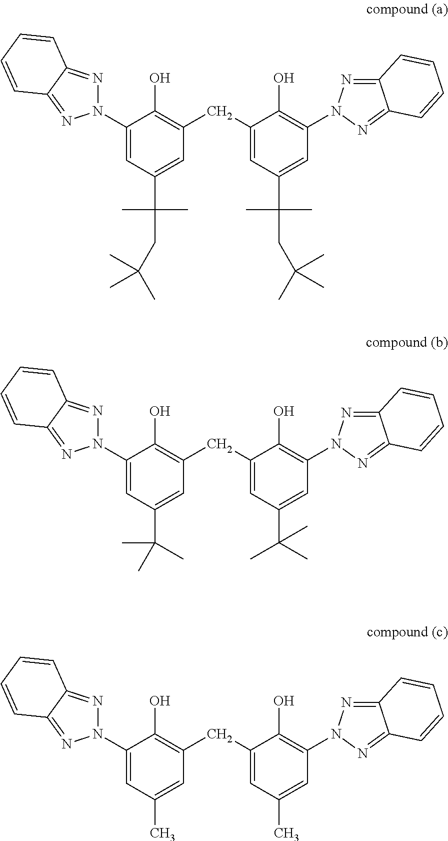 [SMILES: CC(C)(C)CC(C)(C)c1cc(Cc2cc(C(C)(C)CC(C)(C)C)cc(-n3nc4ccccc4n3)c2O)c(O)c(-n2nc3ccccc3n2)c1 ; CC(C)(C)c1cc(Cc2cc(C(C)(C)C)cc(-n3nc4ccccc4n3)c2O)c(O)c(-n2nc3ccccc3n2)c1 ; Cc1cc(Cc2cc(C)cc(-n3nc4ccccc4n3)c2O)c(O)c(-n2nc3ccccc3n2)c1]

Compound (a) of nomenclature 2,2′-methylenebis[6-(2H-benzotriazol-2-yl)-4-(1,1,3,3-tetramethylbutyl)phenol] is in particular sold under the trade name Mixxim BB/200® by the company Fairmount Chemical.

Compound (c) of nomenclature 2,2′-methylenebis[6-(2H-benzotriazol-2-yl)-4-(methyl)phenol] is sold in particular in solid form under the trade name Mixxim BB/200® by the company Fairmount Chemical.

### D/ Vinyl Amides

Among the insoluble organic screening agents of the vinylamide type, mention may be made for example of the compounds having the formula below which are described in application WO 95/22959 (which is an integral part of the content of the description):

T₁₂-(Y)r-C(═O)—C(T₁₃)═C(T₁₄)-N(T₁₅)(T₁₆) (V)

in which T₁₂ is a C₁ to C₁₈, preferably C₁ to C₅, alkyl radical or a phenyl group which is optionally substituted with one, two or three radicals chosen from OH, C₁ to C₁₈ alkyl, C₁ to C₈ alkoxy, or a —C(═O)—OT₁₇ group where T₁₇ is a C₁ to C₁₈ alkyl; T₁₃, T₁₄, T₁₅ and T₁₆, which may be identical or different, denote a C₁ to C₁₈, preferably C₁ to C₅, alkyl radical or a hydrogen atom; Y is N or O and r is 0 or 1.

Among these compounds, mention will more particularly be made of:
4-octylamino-3-penten-2-one;
ethyl 3-octylamino-2-butenoate;
   3-octylamino-1-phenyl-2-buten-1-one;
3-dodecylamino-1-phenyl-2-buten-1-one.

### E/ Cinnamamides

Among the insoluble organic screening agents of the cinnamamide type in accordance with the invention, mention may also be made of the compounds as described in application WO 95/22959 (which forms an integral part of the content of the description) and which correspond to the structure below:

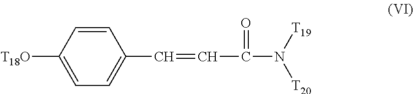

in which OT₁₈ is a hydroxyl or C₁ to C₄ alkoxy radical, preferably methoxy or ethoxy; T₁₉ is hydrogen or C₁ to C₄ alkyl, preferably methyl or ethyl; T₂₀ is a —(CONH)ₛ-phenyl group where s is 0 or 1 and the phenyl group may be substituted with one, two or three groups chosen from OH, C₁ to C₁₈ alkyl, C₁ to C₈ alkoxy, or a —C(═O)—OT₂₁ group where T₂₁ is a C₁ to C₁₈ alkyl and more preferentially T₂₁ is a phenyl, 4-methoxyphenyl or phenylaminocarbonyl group.

Mention may also be made of cinnamamide dimers such as those described in patent U.S. Pat. No. 5,888,481, for instance the compound having the structure:

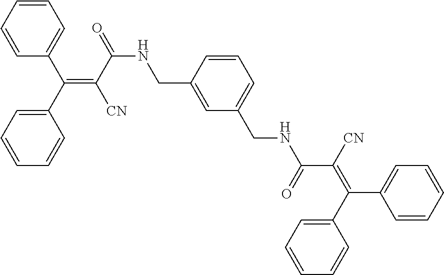 [SMILES: N#CC(C(=O)NCc1cccc(CNC(=O)C(C#N)=C(c2ccccc2)c2ccccc2)c1)=C(c1ccccc1)c1ccccc1]

### F/ Benzazoles

Among the insoluble organic screening agents of the benzazole type, mention may be made of those corresponding to one of the formulae below:

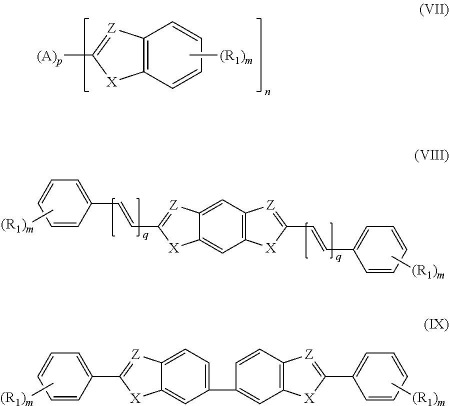

in which each of the symbols X independently represents an oxygen or sulfur atom or a group NR2, each of the symbols Z independently represents a nitrogen atom or a CH group,
each of the symbols R₁ independently represents an OH group, a halogen atom, a linear or branched C₁-C₈ alkyl group, optionally containing a silicon atom, or a linear or branched C₁-C₈ alkoxy group,
each of the numbers m is independently 0, 1 or 2,
n represents an integer between 1 and 4 inclusive,
p is equal to or 1,
each of the numbers q is independently equal to 0 or 1,
each of the symbols R2 independently represents a hydrogen atom, or a benzyl or linear or branched C₁-C₈ alkyl group, optionally containing a silicon atom,
A represents a radical of valency n chosen from those of formulae:

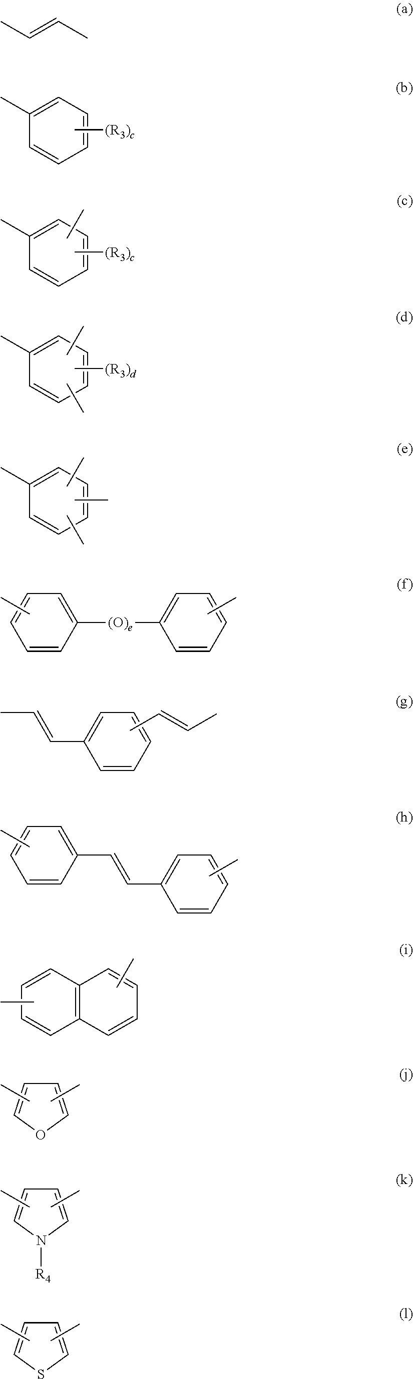

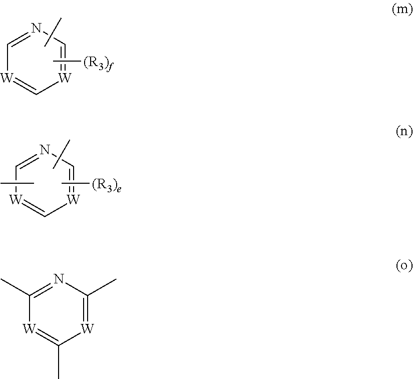

wherein each of the symbols R₃ independently represents a halogen atom or a linear or branched C₁-C₄ alkyl or alkoxy group or a hydroxyl group, and R₄ represents a hydrogen atom or a linear or branched C₁-C₄ alkyl group, c=0-4, d=0-3, e=0 or 1, and f=0-2.

These compounds are in particular described in patents DE 676 103 and CH 350 763, U.S. Pat. No. 5,501,850, U.S. Pat. No. 5,961,960, patent application EP0669323, U.S. Pat. No. 5,518,713, U.S. Pat. No. 2,463,264, the article in J. Am. Chem. Soc., 79, 5706-5708, 1957, the article in J. Am. Chem. Soc., 82, 609-5 611, 1960, patent application EP0921126, and patent application EP712855.

As examples of preferred compounds of formula (VII) of the 2-arylbenzazole family, mention may be made of 2-benzoxazol-2-yl-4-methylphenol, 2-(1H-benzimidazol-2-yl)-4-methoxyphenol or 2-benzothiazol-2-ylphenol, it being possible for these compounds to be prepared for example according to the processes described in patent CH 350 763.

As examples of preferred compounds of formula (VII) of the benzimidazolylbenzazole family, mention will be made of 2,2′-bis-benzimidazole, 5,5′,6,6′-tetramethyl-2,2′-bis-benzimidazole, 5,5′-dimethyl-2,2′-bis-benzimidazole, 6-methoxy-2,2′-bis-benzimidazole, 2-(1H-benzimidazol-2-yl)benzothiazole, 2-(1H-benzimidazol-2-yl)benzoxazole and N,N′-dimethyl-2,2′-bis-benzimidazole, these compounds possibly being prepared according to the procedures described in patents U.S. Pat. No. 5,961,960 and U.S. Pat. No. 2,463,264.

As examples of preferred compounds of formula (VII) of the phenylenebenzazole family, mention will be made of 1,4-phenylene-bis-(2-benzoxazolyl), 1,4-phenylene-bis-(2-benzimidazolyl), 1,3-phenylene-bis-(2-benzoxazolyl), 1,2-phenylene-bis-(2-benzoxazolyl), 1,2-phenylene-bis-(benzimidazolyl), 1,4-phenylene-bis-(N-2-ethylhexyl-2-benzimidazolyl) and 1,4-phenylene-bis-(N-trimethylsilylmethyl-2-benzimidazolyl), these compounds possibly being prepared according to the procedures described in U.S. Pat. No. 2,463,264 and in the publications J. Am. Chem. Soc., 82, 609 (1960) and J. Am. Chem. Soc., 79, 5706-5708 (1957).

As examples of preferred compounds of formula (VII) of the benzofuranyl-benzoxazole family, mention will be made of 2-(2-benzofuranyl)-benzoxazole, 2-(benzofuranyl)-5-methylbenzoxazole and 2-(3-methyl-2-benzofuranyl)-benzoxazole, it being possible for these compounds to be prepared according to the procedures described in U.S. Pat. No. 5,518,713.

As preferred compounds of formula (VIII), mention may be made, for example, of 2,6-diphenyl-1,7-dihydrobenzo[1,2-d; 4,5-d′]diimidazole corresponding to the formula

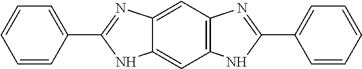 [SMILES: c1ccc(-c2nc3cc4nc(-c5ccccc5)[nH]c4cc3[nH]2)cc1]

or 2,6-distyryl-1,7-dihydrobenzo[1,2-d; 4,5-d′]diimidazole or else 2,6-di(p-tert-butylstyryl)-1,7-dihydrobenzo[1,2-d; 4,5-d′]diimidazole, which may be prepared according to application EP 0 669 323.

As preferred compound of formula (IX), mention may be made of 5,5′-bis-[(phenyl-2)-benzimidazole] having the formula:

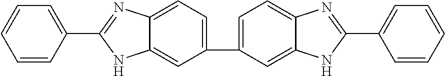 [SMILES: c1ccc(-c2nc3ccc(-c4ccc5nc(-c6ccccc6)[nH]c5c4)cc3[nH]2)cc1]

the preparation of which is described in J. Chim. Phys., 64, 1602 (1967).

Among these insoluble organic compounds which screen out UV radiation, preference is given most particularly to 2-(1H-benzimidazol-2-yl)benzoxazole, 5 ole, 6-methoxy-2,2′-bis-benzimidazole, 2-(1H-benzimidazol-2-yl)benzothiazole, 1,4-phenylenebis-(2-benzoxazolyl), 1,4-phenylene-bis-(2-benzimidazolyl), 1,3-phenylenebis-(2-benzoxazolyl), 1,2-phenylene-bis-(2-benzoxazolyl), 1,2-phenylenebis-(2-benzimidazolyl) and 1,4-phenylene-bis-(N-trimethylsilylmethyl-2-benzimidazolyl).

### G/ Aryl Vinylene Ketones

Among the insoluble organic screening agents of the aryl vinylene ketone type, mention may be made of those corresponding to one of formulae (X) and (XI) below:

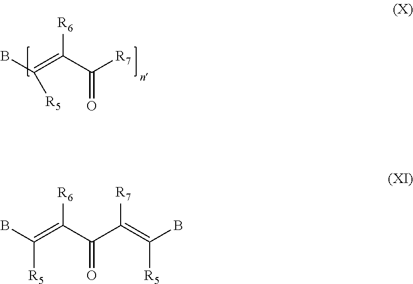

wherein:
n′=1 or 2,
B, in formula (X) when n′=1 or in formula (XI), is an aryl radical chosen from formulae
(a′) to (d′) below, or, in formula (X) when n′=2, is a radical chosen from formulae (e′) to (h′) below:

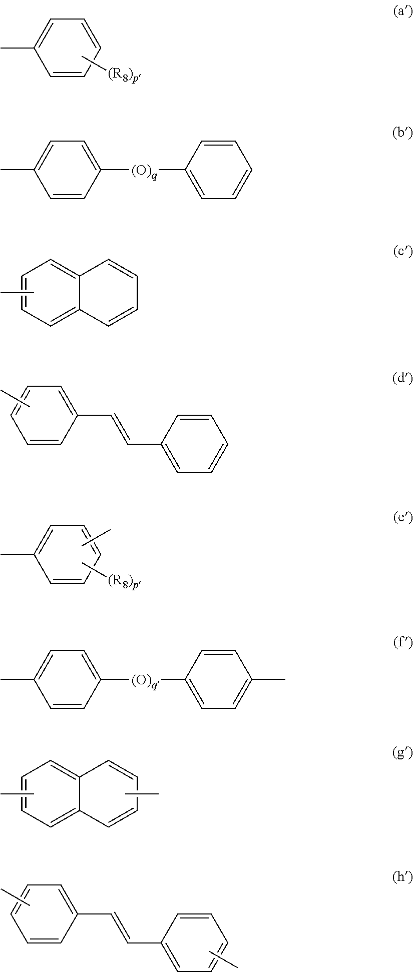

in which:
each of the symbols R₈ independently represents an OH group, a halogen atom, a linear or branched C₁-C₆ alkyl group, optionally containing a silicon atom, a linear or branched C₁-C₆ alkoxy group, optionally containing a silicon atom, a linear or branched C₁-C₅ alkoxycarbonyl group, or a linear or branched C₁-C₆ alkylsulfonamide group, optionally containing a silicon atom or an amino acid function,
p′ represents an integer between 0 and 4 inclusive,
q′ represents 0 or 1,
R₅ represents hydrogen or an OH group,
R₆ represents hydrogen, a linear or branched C₁-C₆ alkyl group, optionally containing a silicon atom, a cyano group, a C₁-C₆ alkylsulfonyl group, or a phenylsulfonyl group,
R₇ represents a linear or branched C₁-C₆ alkyl group, optionally containing a silicon atom, or a phenyl group which can form a bicycle and which is optionally substituted with one or two radicals R₄,
or R₆ and R₇ together form a monocyclic, bicyclic or tricyclic C₂-C₁₀ hydrocarbon-based residue, optionally interrupted with one or more nitrogen, sulfur and oxygen atoms and possibly containing another carbonyl, and optionally substituted with a linear or branched C₁-C₈ alkylsulfonamide group optionally containing a silicon atom or an amino acid function, on the condition that, when n′=1, R₆ and R₇ do not form a camphor nucleus.

As examples of insoluble compounds of formula (X), in which n′=1, which screen out UV radiation and which have a mean particle size of between 10 nm and 5 nm, mention may be made of the following families:
compounds of the styryl ketone type as described in application JP 04 134 042, such as 1-(3,4-dimethoxyphenyl)-4,4-dimethylpent-1-en-3-one:

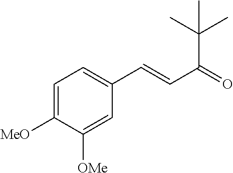 [SMILES: COc1ccc(/C=C/C(=O)C(C)(C)C)cc1OC]

compounds of the benzylidene cineole type such as those described in the article by E. Mariani et al., 16th IFSCC Congress, New York (1990), such as 1,3,3-trimethyl-5-(4-methoxybenzylidene)-2-oxabicyclo[2.2.2]octan-6-one:

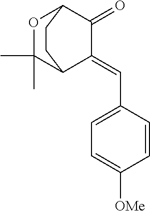 [SMILES: COc1ccc(/C=C2/C(=O)C3CCC2C(C)(C)O3)cc1]

compounds of the benzylidene chromanone type, such as those described in application JP 04 134 043, for instance 3-(4-methoxybenzylidene)-2,3,4a,8a-tetrahydrochromen-4-one:

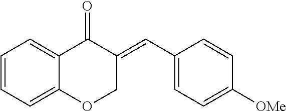 [SMILES: COc1ccc(/C=C2\COc3ccccc3C2=O)cc1]

compounds of the benzylidene thiochromanone type such as those described in application JP 04 134 043, for instance 3-(4-methoxybenzylidene)-2,3,4a,8a-tetrahydrochromen-4-thione:

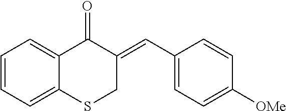 [SMILES: COc1ccc(/C=C2\CSc3ccccc3C2=O)cc1]

compounds of the benzylidene quinuclidinone type such as those described in application EP 0 576 974, for instance 4-methoxybenzylidene-1-azabicyclo[2.2.2]octan-3-one:

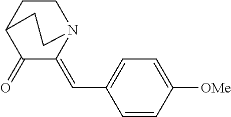 [SMILES: COc1ccc(/C=C2/C(=O)C3CCN2CC3)cc1]

compounds of the benzylidene cycloalcanone type such as those described in application FR 2 395 023, for instance 2-(4-methoxybenzylidene)cyclopentanone and 2-(4-methoxybenzylidene)cyclohexanone:

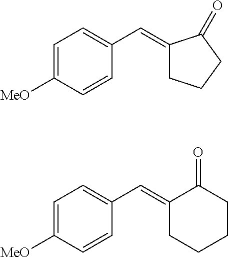 [SMILES: COc1ccc(/C=C2\CCCC2=O)cc1 ; COc1ccc(/C=C2\CCCCC2=O)cc1]

compounds of the benzylidene hydantoin type, such as those described in application JP 01 158 090, for instance 5-(3,4-dimethoxybenzylidene)imidazolidine-2,4-dione:

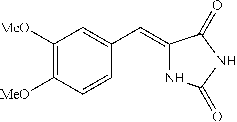 [SMILES: COc1ccc(/C=C2\NC(=O)NC2=O)cc1OC]

compounds of the benzylidene indanone type, such as those described in application JP 04 134 043, for instance 2-(4-methoxybenzylidene)-indan-1-one:

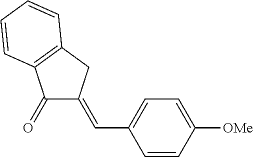 [SMILES: COc1ccc(/C=C2\Cc3ccccc3C2=O)cc1]

compounds of the benzylidene tetralone type, such as those described in application JP 04 134 043, for instance 2-(4-methoxybenzylidene)-3,4-dihydro-2H-naphthalen-1-one:

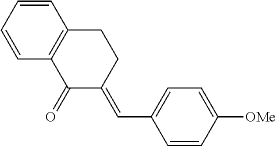 [SMILES: COc1ccc(/C=C2\CCc3ccccc3C2=O)cc1]

compounds of the benzylidene furanone type, such as those described in application EP 0 390 683, for instance 4-(4-methoxybenzylidene)-2,2,5,5-tetramethyldihydrofuran-3-one:

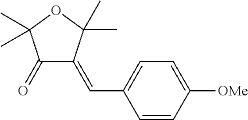 [SMILES: COc1ccc(/C=C2/C(=O)C(C)(C)OC2(C)C)cc1]

compounds of the benzylidene benzofuranone type such as those described in application JP 04 134 041, for instance 2-benzylidenebenzofuran-3-one:

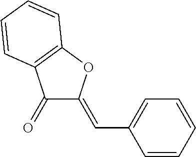 [SMILES: O=C1/C(=C/c2ccccc2)Oc2ccccc21]

compounds of the benzylidene indanedione type such as 2-(3,5-di(tert-butyl)-4-hydroxybenzylidene)indan-1,3-dione:

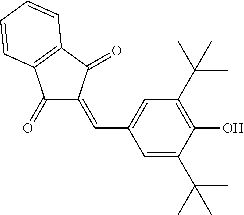 [SMILES: CC(C)(C)c1cc(C=C2C(=O)c3ccccc3C2=O)cc(C(C)(C)C)c1O]

compounds of the benzylidene benzothiofuranone type such as those described in application JP 04,134,043, for instance 2-benzylidenebenzo[b]thiophen-3-one:

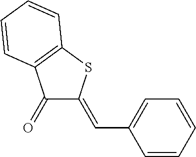 [SMILES: O=C1/C(=C/c2ccccc2)Sc2ccccc21]

compounds of the benzylidene barbiturate type such as 5-(4-methoxybenzylidene)-1,3-dimethylpyrimidine-2,4,6-trione:

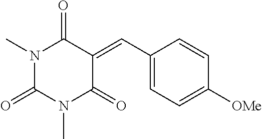 [SMILES: COc1ccc(C=C2C(=O)N(C)C(=O)N(C)C2=O)cc1]

compounds of the benzylidene pyrazolone type such as 4-(4-methoxybenzylidene)-5-methyl-2-phenyl-2,4-dihydropyrazol-3-one:

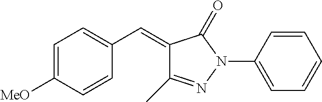 [SMILES: COc1ccc(/C=C2/C(=O)N(c3ccccc3)N=C2C)cc1]

compounds of the benzylidene imidazolone type such as 5-(4-methoxybenzylidene)-2-phenyl-3,5-dihydroimidazol-4-one:

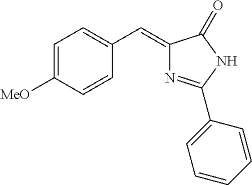 [SMILES: COc1ccc(/C=C2\N=C(c3ccccc3)NC2=O)cc1]

compounds of the chalcone type such as 1-(2-hydroxy-4-methoxyphenyl)-3-phenylpropenone:

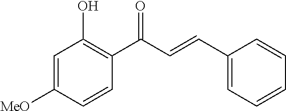 [SMILES: COc1ccc(C(=O)/C=C/c2ccccc2)c(O)c1]

benzylidene one compounds as described in document FR 2 506 1.56, for instance 3-hydroxy-1-(2-hydroxy-4-methoxyphenyl)-3-phenylpropenone:

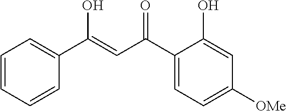 [SMILES: COc1ccc(C(=O)/C=C(\O)c2ccccc2)c(O)c1]

As examples of insoluble compounds of formula (X), in which n′=2, which screen out UV radiation and which have a mean particle size of between 10 nm and 5 μm, mention may be made of the following families:
compounds of the phenylene bis methylidene-nor-camphor type as described in document EP 0 693 471, for instance 1,4-phenylene-bis-{3-methylidenebicyclo[2.2.1]heptan-2-one}:

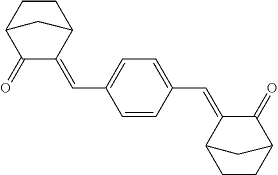 [SMILES: O=C1/C(=C/c2ccc(/C=C3/C(=O)C4CCC3C4)cc2)C2CCC1C2]

compounds of the phenylene bis methylidene camphor type as described in document FR 2 528 420, for instance 1,4-phenylene-bis-{3-methylidene-1,7,7-trimethylbicyclo[2.2.1]heptan-2-one}

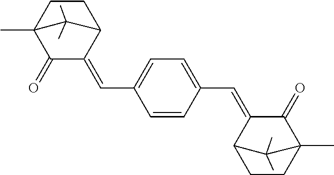

or 1,3-phenylene-bis-{3-methylidene-1,7,7-trimethylbicyclo [2.2.1]heptan-2-one}:

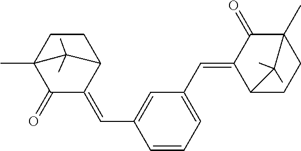

compounds of the phenylene bis methylidene camphor sulfonamide type such as those described in document FR2 529 887, for instance ethyl or 2-ethylhexyl 1,4-phenylene-bis-3,3′-methylidenecamphor-10,10′-sulfonamide:

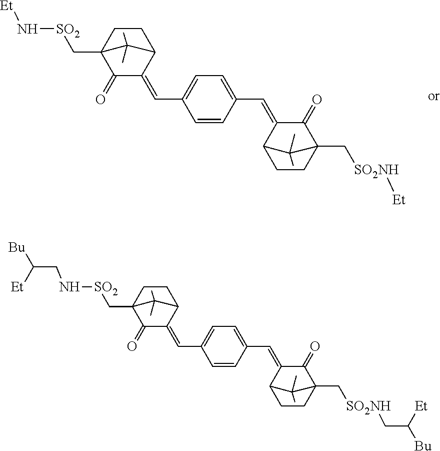

compounds of the phenylene bis methylidene cineole type as described in the article by E. Mariani et al., 16th IFSCC Congress, New York (1990), for instance 1,4-phenylene-bis-{5-methylidene-3,3-dimethyl-2-oxabicyclo[2.2.2]octan-6-one}:

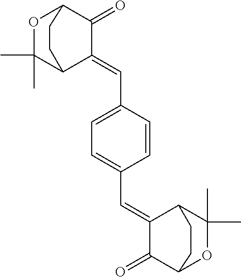

compounds of the phenylene bis methylidene ketotricyclodecane type as described in application EP 0 694 521, for instance 1,4-phenylene-bis-(octahydro-4,7-methano-6-inden-5-one):

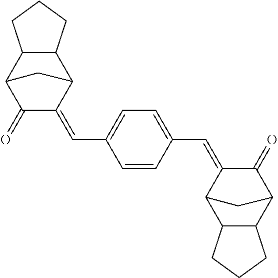 [SMILES: O=C1/C(=C/c2ccc(/C=C3/C(=O)C4CC3C3CCCC43)cc2)C2CC1C1CCCC21]

compounds of the phenylene bis alkylene ketone type such as those described in application JP 04 134 041, for instance 1,4-phenylene-bis-(4,4-dimethyl-pent-1-en-3-one):

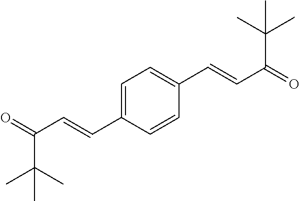 [SMILES: CC(C)(C)C(=O)/C=C/c1ccc(/C=C/C(=O)C(C)(C)C)cc1]

compounds of the phenylene bis methylidene furanone type as described in application FR 2 638 354, for instance 1,4-phenylene-bis-(4-methylidene-2,2,5,5-tetramethyldihydrofuran-3-one):

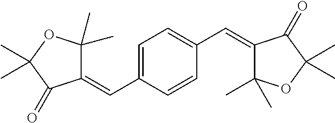

compounds of the phenylene bis methylidene quinuclidinone type such as those described in application EP 0 714 880, for instance 1,4-phenylene-bis-{2-methylidene-1-azabicyclo[2.2.2]octan-3-one}:

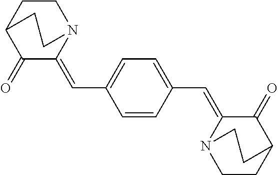 [SMILES: O=C1/C(=C/c2ccc(/C=C3/C(=O)C4CCN3CC4)cc2)N2CCC1CC2]

As compounds of formula (XI), mention may be made of the following families:
compounds of the bis benzylidene cycloalcanone type such as 2,5-dibenzylidenecyclopentanone:

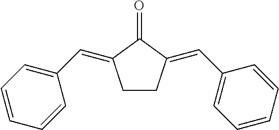 [SMILES: O=C1/C(=C/c2ccccc2)CC/C1=C\c1ccccc1]

compounds of the gamma pyrone type as described in document JP 04 290 882, for instance 2,6-bis-(3,4-dimethoxyphenyl)pyran-4-one:

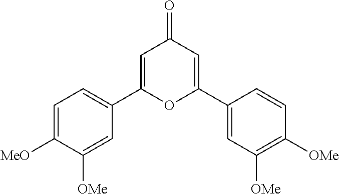 [SMILES: COc1ccc(-c2cc(=O)cc(-c3ccc(OC)c(OC)c3)o2)cc1OC]

Among these insoluble organic compounds which screen out UV radiation, of the aryl vinylene ketone type, preference is given most particularly to the compounds of formula (X) in which n′=2.

### H/ Phenylene Bis-Benzoxazinones

Among the insoluble organic screening agents of the phenylene bis-benzoxazinone type, mention may be made of those corresponding to formula (XII) below:

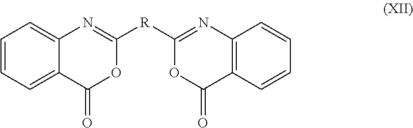

with R representing a divalent aromatic residue chosen from the formulae (e) to (h) below:

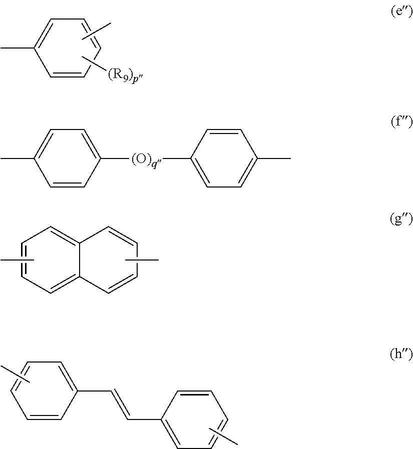

in which:
each of the symbols R₉ independently represents an OH group, a halogen atom, a linear or branched C₁-C₆ alkyl group, optionally containing a silicon atom, a linear or branched C₁-C₆ alkoxy group, optionally containing a silicon atom, a linear or branched C₁-C₅ alkoxycarbonyl group, or a linear or branched C₁-C₆ alkylsulfonamide group, optionally containing a silicon atom or an amino acid function,
p″ represents an integer between 0 and 4 inclusive,
q″ represents 0 or 1.

As examples of insoluble compounds of formula (XII), which screen out UV radiation and which have a mean particle size of between 10 nm and 5 μm, mention may be made of the following derivatives:
2,2′-p-phenylenebis(3,1-benzoxazin-4-one), sold in particular under the trade name Cyasorb UV-3638® by the company Cytec,
2,2′-(4,4′-biphenylene)bis(3,1-benzoxazin-4-one),
2,2′-(2,6-naphthylene)bis(3,1-benzoxazin-4-one).

### I/ Acrylonitrile Amide, Sulfonamide or Carbamate Derivatives

Among the insoluble organic screening agents of the acrylonitrile amide, sulfonamide or carbamate derivative type, mention may be made of those corresponding to formula (XIII) below:

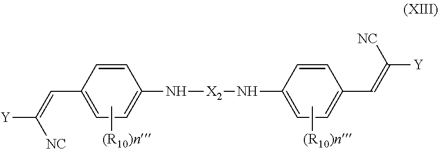

wherein:
R₁₀ represents a linear or branched C₁-C₈ alkyl group,
n′″ is 0, 1 or 2,
X₂ represents a divalent radical of formula —(C═O)—R₁₁—(C═O)—, —SO₂—R₁₁—SO₂— or —(C═O)—O—R₁ₗ—O—(C═O)—,
Y represents a radical —(C═O)—R₁₂ or —SO₂R₁₃,
R₁₁ represents a single bond or a linear or branched C₁-C₃₀ alkylene or C₃-C₃₀ alkenylene divalent radical which may bear one or more hydroxyl substituents and which may contain, in the carbon-based chain, one or more heteroatoms chosen from oxygen, nitrogen and silicon atoms,
R₁₂ represents a radical —OR₁₄ or —NHR₁₄,
R₁₃ represents a linear or branched C₁-C₃₀ alkyl radical, or a phenyl nucleus which is unsubstituted or substituted with C₁-C₄ alkyl or alkoxy radicals,
R₁₄ represents a linear or branched C₁-C₃₀ alkyl or C₃-C₃₀ alkenyl radical which may bear one or more hydroxyl substituents and which may contain, in the carbon-based chain, one or more heteroatoms chosen from oxygen, nitrogen and silicon atoms.

Although, in formula (XIII) above, only the isomers in which the cyano substituent is in the cis position relative to the para-aminophenyl substituent are represented, this formula should be understood as also encompassing the corresponding trans isomers; for each of the two double bonds and independently, the cyano and para-aminophenyl substituents may be in the cis or trans configuration relative to each other.

By way of example, mention may be made of the dimer of 2-ethylhexyl 2-cyano-3-[4-(acetylamino)phenyl]acrylate of formula:

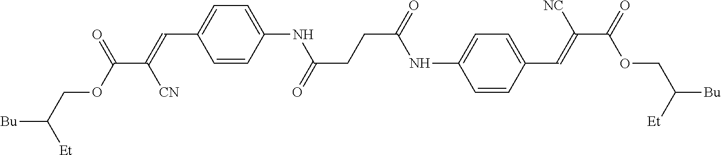

### J/ Polyvalent Metals

Another particular family of insoluble organic screening agents in accordance with the invention are the salts of polyvalent metals (for example Ca²⁺, Zn²⁺, Mg²⁺, Ba²⁺, Al³⁺ or Zr⁴⁺) of sulfonic or carboxylic organic screening agents such as the polyvalent metal salts of sulfonated derivatives of benzylidenecamphor, such as those described in application FR-A 2 639 347; the polyvalent metal salts of sulfonated derivatives of benzimidazole, such as those described in application EP-A-893119; the polyvalent metal salts 5 of cinnamic acid derivatives, such as those described in application JP-87 166 517.

Mention may also be made of the metal, ammonium or substituted-ammonium complexes of UVA and/or UVB organic screening agents as described in patent applications WO93/10753, WO93/11095 and WO95/05150.

Among the insoluble organic UV-screening agents, mention may also be made of the compound 1,1′-(1,4-piperazinediyl)bis[1-[2-[4-(diethylamino)-2-hydroxybenzoyl]phenyl]methanone (CAS 919803-06-8) having the following structure:

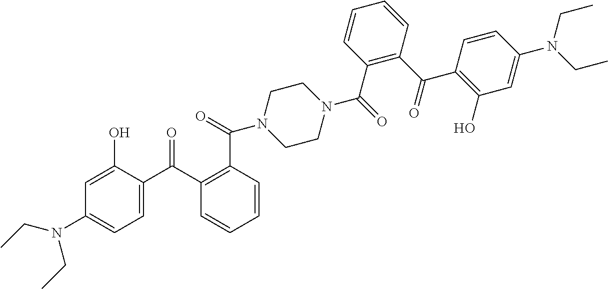 [SMILES: CCN(CC)c1ccc(C(=O)c2ccccc2C(=O)N2CCN(C(=O)c3ccccc3C(=O)c3ccc(N(CC)CC)cc3O)CC2)c(O)c1]

as described in application WO 2007/071 584; this compound advantageously being used in micronized form (mean size of 0.02 to 2 μm), which may be obtained, for example, according to the micronization process described in applications GB-A-2 303 549 and EP-A-893 119, and in particular in the form of an aqueous dispersion.

According to a particularly preferred form of the invention, use will be made of the insoluble organic UV-screening agents chosen from:

(i) symmetrical triazine screening agents substituted with naphthalenyl groups or polyphenyl groups described in U.S. Pat. No. 6,225,467, application WO 2004/085 412 (see compounds 6 and 9) or the document “Symmetrical Triazine Derivatives”, IP.COM IPCOM000031257 Journal, INC West Henrietta, N.Y., US (Sep. 20, 2004), in particular 2,4,6-tris(diphenyl)triazine and 2,4,6-tris(terphenyl)triazine, which is also mentioned in patent applications WO 06/035 000, WO 06/034 982, WO 06/034 991, WO 06/035 007, WO 2006/034 992 and WO 2006/034 985, these compounds advantageously being used in micronized form (mean particle size of 0.02 to 3 μm), which may be obtained, for example, according to the micronization process described in applications GB-A-2 303 549 and EP-A-893 119, and in particular in aqueous dispersion form;
(ii) the methylenebis(hydroxyphenylbenzotriazole) compounds of formula (IV) below:

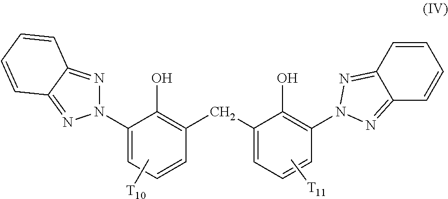

wherein the radicals T₁₀ and T₁₁, which may be identical or different, denote a C₁-C₁₈ alkyl radical which may be substituted with one or more radicals chosen from C₁-C₄ alkyl, C₅-C₁₂ cycloalkyl or an aryl residue;
(iii) and mixtures thereof.

According to a particularly preferred form of the invention, the methylenebis(hydroxyphenylbenzotriazole) compounds of formula (IV) are in the form of an aqueous dispersion of particles having a mean particle size which ranges from 0.01 to 5 μm and more preferentially from 0.01 to 2 μm and more particularly from 0.020 to 2 μm with at least one surfactant of structure CₙH₂ₙ₊₁ O(C₆H₁₀O₅)ₓH in which n is an integer from 8 to 16 and x is the mean degree of polymerization of the unit (C₆H₁₀O₅) and ranges from 1.4 to 1.6 as defined previously. Said surfactant is preferably used at a concentration ranging from 1% to 50% by weight, and more preferentially from 5% to 40% by weight, relative to the benzotriazole screening agent, and the amount of benzotriazole screening agent of formula (I) in the aqueous dispersion preferably ranges from 10% to 50% by weight, and more preferentially from 30 to 50% by weight, relative to the total weight of the dispersion.

The mean particle diameter is measured using a particle size distribution analyzer of the Culter N4 PLUS® type manufactured by Beckman Coulter Inc.

According to a particularly preferred form of the invention, the methylenebis(hydroxyphenylbenzotriazole) compounds of formula (IV) may be in the form of an aqueous dispersion of particles having a mean particle size which ranges from 0.02 to 2 μm and more preferentially from 0.01 to 1.5 μm and more particularly from 0.02 to 1 μm in the presence of at least one polyglycerol mono(C₈-C₂₀)alkyl ester having a degree of glycerol polymerization of at least 5, such as the aqueous dispersions described in application WO2009/063392.

As an example of surfactants which are polyglycerol mono(C₈-C₂₀)alkyl esters, mention may be made of decaglyceryl caprate, decaglyceryl laurate, decaglyceryl myristate, decaglyceryl oleate, decaglyceryl stearate, decaglyceryl isostearate, hexaglyceryl caprate, hexaglyceryl laurate, hexaglyceryl myristate, hexaglyceryl oleate, hexaglyceryl stearate, hexaglyceryl isostearate, pentaglyceryl caprate, pentaglyceryl laurate, pentaglyceryl myristate, pentaglyceryl oleate, pentaglyceryl stearate, and pentaglyceryl isostearate.

Use will more particularly be made of:
decaglyceryl caprate such as the products sold under the following trade names: Sunsoft Q10Y®, Sunsoft Q10S®, Sunsoft Q12Y®, Sunsoft Q12S®, Sunsoft M12J® by the company Taiyo Kagaku Co. Ltd., Nikkol Decaglyn 1-L by the company Nikko Chemicals Co. Ltd, Ryoto-Polyglycerylester L-10D® and L-7D® by the company Mitsubishi-Kagaku Co. Ltd.,
decaglyceryl laurate such as the products sold under the following trade names: Sunsoft Q14Y®, Sunsoft Q14S®, Sunsoft Q12Y®, Sunsoft Q12S®, Sunsoft M12J® by the company Taiyo Kagaku Co. Ltd., Nikkol Decaglyn 1-M® by the company Nikko Chemicals Co. Ltd, Ryoto-Polyglycerylester M-10D and M-7D by the company Mitsubishi-Kagaku Co. Ltd.,
decaglyceryl stearate such as the products sold under the following trade names: Sunsoft Q18Y®, Sunsoft Q18S®, Sunsoft Q12Y®, Sunsoft Q12S®, Sunsoft M12J® by the company Taiyo Kagaku Co. Ltd., Nikkol Decaglyn 1-SV by the company Nikko Chemicals Co. Ltd, Ryoto-Polyglycerylester S-15D by the company Mitsubishi-Kagaku Co. Ltd.,
hexaglyceryl caprate such as the products sold under the following trade names: Nikkol Hexaglyn 1-L® by the company Nikko Chemicals Co. Ltd, Glysurf 6ML by the company Aoki Oil Industrial Co. Ltd., Unigly GL-106® by the company Nippon Oil & Fats Co. Ltd.,
hexaglyceryl myristate such as the products sold under the following trade names: Nikkol Hexaglyn 1-M®, Nikkol Hexaglyn 1-OV® by the company Nikko Chemicals Co. Ltd, Glysurf 6ML® by the company Aoki Oil Industrial Co. Ltd., Unigly GL-106 by the company Nippon Oil & Fats Co. Ltd.,
hexaglyceryl stearate such as the products sold under the following trade names: Nikkol Hexaglyn 1-M®, Nikkol Hexaglyn 1-SV® by the company Nikko Chemicals Co. Ltd, EmalexMSG-6K® by the company Nihon-Emulsion Co. Ltd., Unigly GL-106 by the company Nippon Oil & Fats Co. Ltd.,
hexaglyceryl isostearate such as the products sold under the following trade names: Matsumate MI-610® by the company Matsumoto Fine Chemical Co. Ltd,
pentaglyceryl caprate such as the products sold under the following trade names: Sunsoft A10E®, by the company Taiyo Kagaku Co. Ltd.,
pentaglyceryl laurate such as the products sold under the following trade names: Sunsoft A12E®, Sunsoft A121E® by the company Taiyo Kagaku Co. Ltd.,
pentaglyceryl myristate such as the products sold under the following trade names: Sunsoft A14E®, Sunsoft A141E® by the company Taiyo Kagaku Co. Ltd.,
pentaglyceryl oleate such as the products sold under the following trade names: Sunsoft A17E®, Sunsoft A171E® by the company Taiyo Kagaku Co. Ltd.,
pentaglyceryl stearate such as the products sold under the following trade names: Sunsoft A18E®, Sunsoft A181E by the company Taiyo Kagaku Co. Ltd.

Among these surfactants, those having an HLB greater than or equal to 14.5, and more preferentially greater than or equal to 15, are preferably used. As examples of surfactants which are mono-(C₈-C₂₀)alkyl esters of polyglycerol having a degree of polymerization having a degree of glycerol polymerization of at least 5 and having an HLB greater than or equal to 14.5, mention may be made of decaglyceryl caprate, decaglyceryl laurate, decaglyceryl myristate, decaglyceryl oleate, decaglyceryl stearate, decaglyceryl isostearate, hexaglyceryl laurate, pentaglyceryl caprate, pentaglyceryl laurate, pentaglyceryl myristate, pentaglyceryl oleate, and pentaglyceryl stearate. As examples of surfactants which are mono-(C₈-C₂₀)alkyl esters of polyglycerol having a degree of polymerization having a degree of glycerol polymerization of at least 5 and having an HLB greater than or equal to 15, mention may be made of decaglyceryl caprate and decaglyceryl laurate.

The amount of methylenebis(hydroxyphenylbenzotriazole) compound of formula (IV) in the aqueous dispersion preferably ranges from 10% to 50% by weight, and more preferentially from 30% to 50% by weight, relative to the total weight of the dispersion.

Preferentially, the methylenebis(hydroxyphenylbenzotriazole) compound/mono-(C₈-C₂₀)alkyl ester of polyglycerol weight ratio ranges from 0.05 to 0.5, and more preferentially from 0.1 to 0.3.

In these aqueous dispersions, use will preferentially be made, as methylenebis(hydroxyphenylbenzotriazole) compound of formula (IV), of the compound 2,2′-methylenebis[6-(2H-benzotriazol-2-yl)-4-(1,1,3,3-tetramethylbutyl)phenol] having the structure:

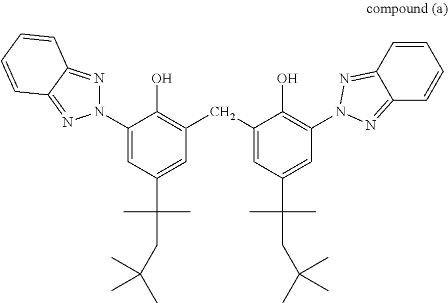 [SMILES: CC(C)(C)CC(C)(C)c1cc(Cc2cc(C(C)(C)CC(C)(C)C)cc(-n3nc4ccccc4n3)c2O)c(O)c(-n2nc3ccccc3n2)c1]

such as the commercial product sold under the name Tinosorb M® by BASF which is an aqueous dispersion comprising decylglucoside, xanthan gum and propylene glycol (INCI name: Methylene Bis-Benzotriazolyl Tetramethylbutylphenol (and) Aqua (and) Decyl Glucoside (and) Propylene Glycol (and) Xanthan Gum).

### IV/ Inorganic UV-Screening Agents

The inorganic UV-screening agents used in accordance with the present invention are metal oxide pigments. More preferentially, the inorganic UV-screening agents of the invention are metal oxide particles with a mean elementary particle size of less than or equal to 0.5 μm, more preferentially between 0.005 and 0.5 μm, even more preferentially between 0.01 and 0.2 μm, better still between 0.01 and 0.1 μm and more particularly preferentially between 0.015 and 0.05 μm.

The term “mean elementary size” is intended to mean the size of non-aggregated particles.

They may be chosen in particular from titanium oxide, zinc oxide, iron oxide, zirconium oxide and cerium oxide, or mixtures thereof.

Such coated or uncoated metal oxide pigments are described in particular in patent application EP-A-0 518 773. Commercial pigments that may be mentioned include in particular the products sold by the companies Sachtleben Pigments, Tayca, Merck and Degussa.

The metal oxide pigments may be coated or uncoated.

The coated pigments are pigments that have undergone one or more surface treatments of chemical, electronic, mechanochemical and/or mechanical nature with compounds such as amino acids, beeswax, fatty acids, fatty alcohols, anionic surfactants, lecithins, sodium, potassium, zinc, iron or aluminum salts of fatty acids, metal alkoxides (of titanium or aluminum), polyethylene, silicones, proteins (collagen, elastin), alkanolamines, silicon oxides, metal oxides or sodium hexametaphosphate.

The coated pigments are more particularly titanium oxides that have been coated:
with silica, such as the product Sunveil from the company Ikeda,
with silica and iron oxide, such as the product Sunveil F from the company Ikeda,
with silica and alumina, such as the products Microtitanium Dioxide MT 500 SA and Microtitanium Dioxide MT 100 SA from the company Tayca and Tioveil from the company Tioxide,
with alumina, such as the products Tipaque TTO-55 (B) and Tipaque TTO-55 (A) from the company Ishihara, and UVT 14/4 from the company Sachtleben Pigments,
with alumina and aluminum stearate, such as the products Microtitanium Dioxide MT 100 T, MT 100 TX, MT 100 Z and MT-01 from the company Tayca, the products Solaveil CT-10 W and Solaveil CT 100 from the company Uniqema and the product Eusolex T-AVO from the company Merck,
with silica, alumina and alginic acid, such as the product MT-100 AQ from the company Tayca,
with alumina and aluminum laurate, such as the product Microtitanium Dioxide MT 100 S from the company Tayca,
with iron oxide and iron stearate, such as the product Microtitanium Dioxide MT 100 F from the company Tayca,
with zinc oxide and zinc stearate, such as the product BR 351 from the company Tayca,
with silica and alumina and treated with a silicone, such as the products Microtitanium Dioxide MT 600 SAS, Microtitanium Dioxide MT 500 SAS or Microtitanium Dioxide MT 100 SAS from the company Tayca,
with silica, alumina and aluminum stearate and treated with a silicone, such as the product STT-30-DS from the company Titan Kogyo,
with silica and treated with a silicone, such as the product UV-Titan X 195 from the company Sachtleben Pigments,
with alumina and treated with a silicone, such as the products Tipaque TTO-55 (S) from the company Ishihara or UV Titan M 262 from the company Sachtleben Pigments,
with triethanolamine, such as the product STT-65-S from the company Titan Kogyo,
with stearic acid, such as the product Tipaque TTO-55 (C) from the company Ishihara,
with sodium hexametaphosphate, such as the product Microtitanium Dioxide MT 150 W from the company Tayca,
TiO₂ treated with octyltrimethylsilane, sold in particular under the trade name T 805 by the company Degussa Silices,
TiO₂ treated with a polydimethylsiloxane, sold in particular under the trade name 70250 Cardre UF TiO2SI3 by the company Cardre,
anatase/rutile TiO₂ treated with a polydimethylhydrogenosiloxane, sold in particular under the trade name Microtitanium Dioxide USP Grade Hydrophobic by the company Color Techniques.

Mention may also be made of TiO₂ pigments doped with at least one transition metal such as iron, zinc or manganese and more particularly manganese. Preferably, said doped pigments are in the form of an oily dispersion. The oil present in the oily dispersion is preferably chosen from triglycerides including those of capric/caprylic acids. The oily dispersion of titanium oxide particles may also comprise one or more dispersants, for instance a sorbitan ester, for instance sorbitan isostearate, or a polyoxyalkylenated fatty acid ester of glycerol, for instance TRI-PPG-3 myristyl ether citrate and polyglyceryl-3 polyricinoleate. Preferably, the oily dispersion of titanium oxide particles comprises at least one dispersant chosen from polyoxyalkylenated fatty acid esters of glycerol. Mention may be made more particularly of the oily dispersion of TiO₂ particles doped with manganese in capric/caprylic acid triglyceride in the presence of TRI-PPG-3 myristyl ether citrate and polyglyceryl-3 polyricinoleate and sorbitan isostearate having the INCI name: titanium dioxide (and) TRI-PPG-3 myristyl ether citrate (and) polyglyceryl-3 ricinoleate (and) sorbitan isostearate, for instance the product sold in particular under the trade name Optisol TD50 by the company Croda.

The uncoated titanium oxide pigments are sold, for example, by the company Tayca under the trade names Microtitanium Dioxide MT 500 B or Microtitanium Dioxide MT 600 B, by the company Degussa under the name P 25, by the company Wachker under the name Transparent titanium oxide PW, by the company Miyoshi Kasei under the name UFTR, by the company Tomen under the name ITS and by the company Tioxide under the name Tioveil AQ.

The uncoated zinc oxide pigments are for example:
those sold in particular under the name Z-Cote by the company Sunsmart;
those sold in particular under the name Nanox by the company Elementis;
those sold in particular under the name Nanogard WCD 2025 by the company Nanophase Technologies.

The coated zinc oxide pigments are for example:
those sold in particular under the name Zinc Oxide CS-5 by the company Toshibi (ZnO coated with polymethylhydrosiloxane);
those sold in particular under the name Nanogard Zinc Oxide FN by the company Nanophase Technologies (as a 40% dispersion in Finsolv TN, C₁₂-C₁₅ alkyl benzoate);
those sold in particular under the names Daitopersion Zn-30 and Daitopersion Zn-50 by the company Daito (dispersions in cyclopolymethylsiloxane/oxyethylenated polydimethylsiloxane, containing 30% or 50% of zinc oxides coated with silica and polymethylhydrosiloxane);
those sold in particular under the name NFD Ultrafine ZnO by the company Daikin (ZnO coated with perfluoroalkyl phosphate and copolymer based on perfluoroalkylethyl as a dispersion in cyclopentasiloxane);
those sold in particular under the name SPD-Z1 by the company Shin-Etsu (ZnO coated with silicone-grafted acrylic polymer, dispersed in cyclodimethylsiloxane);
those sold in particular under the name Escalol Z100 by the company ISP (alumina-treated ZnO dispersed in an ethylhexyl methoxycinnamate/PVP-hexadecene copolymer/methicone mixture);
those sold in particular under the name Fuji ZnO-SMS-10 by the company Fuji Pigment (ZnO coated with silica and polymethylsilsesquioxane);
those sold in particular under the name Nanox Gel TN by the company Elementis (ZnO dispersed at a concentration of 55% in C₁₂-C₁₅ alkyl benzoate with hydroxystearic acid polycondensate).

The uncoated cerium oxide pigments may be, for example, those sold under the name Colloidal Cerium Oxide by the company Rhône-Poulenc.

The uncoated iron oxide pigments are sold, for example, by the company Arnaud under the names Nanogard WCD 2002 (FE 45B), Nanogard Iron FE 45 BL AQ, Nanogard FE 45R AQ and Nanogard WCD 2006 (FE 45R) or by the company Mitsubishi under the name TY-220.
The coated iron oxide pigments are sold, for example, by the company Arnaud under the names Nanogard WCD 2008 (FE 45B FN), Nanogard WCD 2009 (FE 45B 556), Nanogard FE 45 BL 345 and Nanogard FE 45 BL or by the company BASF under the name Transparent Iron Oxide.
Mention may also be made of mixtures of metal oxides, in particular of titanium dioxide and of cerium dioxide, including the equal-weight mixture of titanium dioxide and cerium dioxide coated with silica, sold by the company Ikeda under the name Sunveil A, and also the mixture of titanium dioxide and zinc dioxide coated with alumina, silica and silicone, such as the product M 261 sold by the company Sachtleben Pigments, or coated with alumina, silica and glycerol, such as the product M 211 sold by the company Sachtleben Pigments.
According to the invention, coated or uncoated titanium oxide pigments are particularly preferred.

As outlined above, according to a particularly preferred embodiment, the UV-screening agent(s) are chosen from water-soluble organic UV-screening agents, liposoluble organic UV-screening agents, and mixtures thereof.

Preferably, the liposoluble organic UV-screening agents are chosen from dibenzoylmethane compounds, salicylic compounds, β,β-diphenylacrylate compounds, benzophenone compounds, phenyl benzotriazole compounds, triazine compounds, and mixtures thereof, more preferentially chosen from butyl methoxydibenzoylmethane, ethylhexyl salicylate, octocrylene, n-hexyl 2-(4-diethylamino-2-hydroxybenzoyl)benzoate, drometrizole trisiloxane, bis-ethylhexyloxyphenol methoxyphenyl triazine, ethylhexyl triazone, diethylhexyl butamido triazone, and mixtures thereof, and even more preferentially chosen from butyl methoxydibenzoylmethane, ethylhexyl salicylate, octocrylene, and mixtures thereof.

Preferably, the water-soluble organic UV-screening agents are chosen from benzylidenecamphor compounds, phenyl benzimidazole compounds, and mixtures thereof, and more preferentially chosen from phenylbenzimidazole sulfonic acid, terephthalylidene dicamphor sulfonic acid, and mixtures thereof.

The UV-screening agents suitable for use in the invention are present in amounts ranging from 0.1% to 40% by weight, preferably ranging from 1% to 20% by weight, and more preferentially ranging from 5% to 20% by weight relative to the total weight of the composition.

Physiologically Acceptable Medium

As presented above, a composition according to the invention may advantageously be a cosmetic or dermatological composition.

In this particular embodiment, since a composition according to the invention is intended for topical application to the skin and/or the nails, it contains a physiologically acceptable medium.

For the purposes of the present invention, the term “physiologically acceptable medium” is intended to mean a medium that is compatible with the skin and/or the nails.

Thus, the physiologically acceptable medium is in particular a cosmetically or dermatologically acceptable medium, i.e. a medium that has no unpleasant odor, color or appearance, and that does not cause the user any unacceptable stinging, tautness or redness.

Aqueous Phase

The aqueous phase of a composition according to the invention comprises water and optionally a water-soluble solvent.

In the present invention, the term “water-soluble solvent” denotes a compound that is liquid at ambient temperature and water-miscible (miscibility with water of greater than 50% by weight at 25° C. and atmospheric pressure).

The water-soluble solvents that may be used in the composition of the invention may also be volatile.

Among the water-soluble solvents that may be used in the composition in accordance with the invention, mention may be made in particular of lower monoalcohols containing from 1 to 5 carbon atoms such as ethanol and isopropanol, glycols containing from 2 to 8 carbon atoms such as ethylene glycol, propylene glycol, 1,3-butylene glycol and dipropylene glycol, C₃ and C₄ ketones and C₂-C₄ aldehydes.

The aqueous phase (water and optionally the water-miscible solvent) may be present in the composition in a content ranging from 5% to 95%, better still from 30% to 80% by weight and preferably from 40% to 75% by weight relative to the total weight of said composition.

According to another embodiment variant, the aqueous phase of a composition according to the invention may comprise at least one C₂-C₃₂ polyol.

For the purposes of the present invention, the term “polyol” should be understood as meaning any organic molecule comprising at least two free hydroxyl groups.

Preferably, a polyol in accordance with the present invention is present in liquid form at ambient temperature.

A polyol that is suitable for use in the invention may be a compound of linear, branched or cyclic, saturated or unsaturated alkyl type, bearing on the alkyl chain at least two —OH functions, in particular at least three —OH functions and more particularly at least four —OH functions.

The polyols that are advantageously suitable for formulating a composition according to the present invention are those in particular containing from 2 to 32 carbon atoms and preferably 3 to 16 carbon atoms.

Advantageously, the polyol may be chosen, for example, from ethylene glycol, pentaerythritol, trimethylolpropane, propylene glycol, 1,3-propanediol, butylene glycol, isoprene glycol, pentylene glycol, hexylene glycol, glycerol, polyglycerols such as glycerol oligomers, for instance diglycerol, and polyethylene glycols, and mixtures thereof.

According to a preferred embodiment of the invention, said polyol is chosen from ethylene glycol, pentaerythritol, trimethylolpropane, propylene glycol, glycerol, polyglycerols, polyethylene glycols and mixtures thereof.

According to a particular mode, the composition of the invention may comprise at least propylene glycol.

According to another particular mode, the composition of the invention may comprise at least glycerol.

Depending on the presentation form or, when the composition is in the form of an emulsion, depending on the sense of the emulsion, the aqueous phase may be composed of a synthetic phyllosilicate that is suitable for use in the invention in gel form, alone or in combination with other gelling agents.

As presented above, according to a particular embodiment, a synthetic phyllosilicate that is suitable for use in the invention may be used in the form of an aqueous or aqueous-alcoholic gel. When the gel is aqueous, it may then constitute all or part of the aqueous phase. In this respect, it acts as a rheological agent, an agent for stabilizing the emulsion. Thus, the stability of the final composition is improved. This property also applies when the synthetic phyllosilicate gel suitable for use in the invention is used in combination with other aqueous gelling agents.

According to one particular embodiment, a synthetic phyllosilicate suitable for use in the invention in aqueous gel or aqueous-alcoholic gel form constitutes the aqueous phase of a composition according to the invention, i.e. the aqueous phase of the composition is exclusively constituted of this gel.

Fatty Phase

For the purposes of the invention, the fatty phase includes any liquid fatty substance, generally oils (also known as liquid or oily fatty phase), or solid fatty substance like waxes or pasty compounds (also known as solid fatty phase).

For the purposes of the invention, a liquid fatty phase comprises at least one oil.

The term “oil” is intended to mean any fatty substance that is in liquid form at ambient temperature and atmospheric pressure.

An oily phase that is suitable for preparing the cosmetic compositions according to the invention may comprise hydrocarbon-based oils, silicone oils, fluoro oils or non-fluoro oils, or mixtures thereof.

The oils may be volatile or nonvolatile.

They may be of animal, plant, mineral or synthetic origin. According to one implementation variant, oils of plant origin are preferred.

For the purposes of the present invention, the term “nonvolatile oil” is intended to mean an oil with a vapor pressure of less than 0.13 Pa.

For the purposes of the present invention, the term “silicone oil” is intended to mean an oil comprising at least one silicon atom, and in particular at least one Si—O group.

The term “fluoro oil” is intended to mean an oil comprising at least one fluorine atom.

The term “hydrocarbon-based oil” is intended to mean an oil mainly containing hydrogen and carbon atoms.

The oils may optionally comprise oxygen, nitrogen, sulfur and/or phosphorus atoms, for example in the form of hydroxyl or acid radicals.

For the purposes of the invention, the term “volatile oil” is intended to mean any oil that is capable of evaporating on contact with the skin in less than one hour, at ambient temperature and atmospheric pressure. The volatile oil is a volatile cosmetic compound, which is liquid at ambient temperature, in particular having a nonzero vapor pressure, at ambient temperature and atmospheric pressure, in particular having a vapor pressure ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

Volatile Oils

The volatile oils may be hydrocarbon-based oils or silicone oils.

Among the volatile hydrocarbon-based oils containing from 8 to 16 carbon atoms, mention may be made in particular of branched C₈-C₁₆ alkanes, for instance C₈-C₁₆ isoalkanes (also known as isoparaffins), isododecane, isodecane, isohexadecane and, for example, the oils sold under the trade names Isopar or Permethyl, branched C₈-C₁₆ esters, for instance isohexyl neopentanoate, and mixtures thereof. Preferably, the volatile hydrocarbon-based oil is selected from volatile hydrocarbon-based oils containing from 8 to 16 carbon atoms, and mixtures thereof, in particular from isododecane, isodecane and isohexadecane, and is in particular isohexadecane.

Mention may also be made of volatile linear alkanes comprising from 8 to 16 carbon atoms, in particular from 10 to 15 carbon atoms and more particularly from 11 to 13 carbon atoms, for instance n-dodecane (C₁₂) and n-tetradecane (C₁₄) sold by Sasol under the respective references Parafol 12-97 and Parafol 14-97, and also mixtures thereof, the undecane-tridecane mixture, mixtures of n-undecane (C₁₁) and of n-tridecane (C₁₃) obtained in Examples 1 and 2 of application WO 2008/155 059 from the company Cognis, and mixtures thereof.

Volatile silicone oils that may be mentioned include linear volatile silicone oils such as hexamethyldisiloxane, octamethyltrisiloxane, decamethyltetrasiloxane, tetradecamethylhexasiloxane, hexadecamethylheptasiloxane and dodecamethylpentasiloxane.

Volatile cyclic silicone oils that may be mentioned include hexamethylcyclotrisiloxane, octamethylcyclotetrasiloxane, decamethylcyclopentasiloxane and dodecamethylcyclohexasiloxane.

Nonvolatile Oils

The nonvolatile oils may, in particular, be chosen from nonvolatile hydrocarbon-based, fluoro and/or silicone oils.

Nonvolatile hydrocarbon-based oils that may in particular be mentioned include:
hydrocarbon-based oils of animal origin,
hydrocarbon-based oils of plant origin, synthetic ethers containing from 10 to 40 carbon atoms, such as dicaprylyl ether,
synthetic esters, such as the oils having formula R₁COOR₂, in which R₁ represents a linear or branched fatty acid residue comprising from 1 to 40 carbon atoms and R₂ represents a hydrocarbon-based chain, which is in particular branched, containing from 1 to 40 carbon atoms, on condition that R₁+R₂ is ≧10. The esters may be chosen in particular from fatty acid and fatty alcohol esters, such as for example cetostearyl octanoate, isopropyl alcohol esters such as isopropyl myristate or isopropyl palmitate, ethyl palmitate, 2-ethylhexyl palmitate, isopropyl stearate, octyl stearate, hydroxylated esters, such as isostearyl lactate or octyl hydroxystearate, alkyl or polyalkyl ricinoleates, hexyl laurate, neopentanoic acid esters, such as isodecyl neopentanoate or isotridecyl neopentanoate, and isononanoic acid esters, such as isononyl isononanoate or isotridecyl isononanoate,
polyol esters and pentaerythritol esters, such as dipentaerythrityl tetrahydroxystearate/tetraisostearate,
fatty alcohols that are liquid at ambient temperature, with a branched and/or unsaturated carbon-based chain containing from 12 to 26 carbon atoms, for instance 2-octyldodecanol, isostearyl alcohol and oleyl alcohol,
C₁₂-C₂₂ higher fatty acids, such as oleic acid, linoleic acid, linolenic acid, and mixtures thereof,
non-phenyl silicone oils, for instance caprylyl methicone, and
phenyl silicone oils, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates, dimethicones or phenyl trimethicone with a viscosity of less than or equal to 100 cSt, and trimethyl-pentaphenyl-trisiloxane, and mixtures thereof; and also mixtures of these various oils.

Preferably, a composition according to the invention comprises volatile and/or nonvolatile silicone oils.

A composition according to the invention may comprise from 5% to 95% by weight, better still from 5% to 40% by weight and preferably from 7% to 35% by weight of oil(s) relative to the total weight of said composition.

As mentioned above, the oily phase according to the invention may have a threshold stress of greater than 1.5 Pa and preferably greater than 10 Pa. This threshold stress value reflects a gel-type texture of this oily phase.

Waxes

For the purposes of the present invention, the term “wax” is intended to mean a lipophilic fatty compound that is solid at ambient temperature (25° C.), with a reversible solid/liquid change of state, having a melting point of greater than 30° C. which may be up to 200° C., a hardness of greater than 0.5 MPa, and having an anisotropic crystal organization in the solid state. By bringing the wax to its melting point, it is possible to make it miscible with oils and to form a microscopically homogeneous mixture, but on returning the temperature of the mixture to ambient temperature, recrystallization of the wax in the oils of the mixture is obtained.

The waxes that may be used in the invention are compounds that are solid at ambient temperature, which are intended to structure the composition, in particular in stick form; they may be hydrocarbon-based, fluoro- and/or silicone-based and may be of plant, mineral, animal and/or synthetic origin. In particular, they have a melting point of greater than 40° C. and better still greater than 45° C.

As wax that may be used in the invention, mention may be made of those generally used in cosmetics: they are in particular of natural origin, such as beeswax, carnauba wax, candelilla wax, ouricury wax, Japan wax, cork fiber wax or sugarcane wax, rice wax, montan wax, paraffin, lignite wax or microcrystalline wax, ceresin or ozokerite, hydrogenated oils such as jojoba oil; synthetic waxes such as polyethylene waxes derived from the polymerization or copolymerization of ethylene and Fischer-Tropsch waxes, or alternatively fatty acid esters such as octacosanyl stearate, glycerides that are solid at 40° C. and better still at 45° C., silicone waxes such as alkyl or alkoxy dimethicones with an alkyl or alkoxy chain of 10 to 45 carbon atoms, poly(di)methylsiloxane esters that are solid at 40° C., the ester chain of which comprises at least 10 carbon atoms; and mixtures thereof.

As a guide, a composition according to the invention may comprise from 0.01% to 50%, preferably from 2% to 40% and better still from 5% to 30% by weight of wax(es), relative to the total weight of the composition.

Pasty Compound

For the purposes of the present invention, the term “pasty” is intended to denote a lipophilic fatty compound with a reversible solid/liquid change of state, and comprising at a temperature of 23° C. a liquid fraction and a solid fraction.

The pasty compound is advantageously chosen from:
lanolin and derivatives thereof,
polymeric or non-polymeric fluoro compounds,
polymeric or non-polymeric silicone compounds,
vinyl polymers, in particular:
olefin homopolymers,
olefin copolymers,
hydrogenated diene homopolymers and copolymers;
linear or branched homopolymer or copolymer oligomers of alkyl (meth)acrylates preferably bearing a C₈-C₃₀ alkyl group,
homopolymer and copolymer oligomers of vinyl esters bearing C₈-C₃₀ alkyl groups,
homopolymer and copolymer oligomers of vinyl ethers bearing C₈-C₃₀ alkyl groups,
liposoluble polyethers resulting from polyetherification between one or more C₂-C₁₀₀ and preferably C₂-C₅₀ diols,
esters,
polyvinyl laurate; and
mixtures thereof.

As a guide, a composition according to the invention may comprise from 1% to 99%, preferably from 1% to 60%, better from 2% to 30% and better still from 5% to 20% by weight of pasty compound(s), relative to the total weight of the composition.

Other fatty substances may also be present in the oily phase, namely, for example, fatty acids comprising from 8 to 30 carbon atoms, for instance stearic acid, lauric acid or palmitic acid; fatty alcohols comprising from 8 to 30 carbon atoms, for instance stearyl alcohol or cetyl alcohol and mixtures thereof (cetearyl alcohol).

The liquid fatty phase may [contain] other compounds dissolved in the oils, such as gelling agents and/or structuring agents. These compounds may be chosen in particular from gums, such as silicone gums (dimethiconol); silicone resins, such as trifluoromethyl(C1-C4 alkyl) dimethicone and trifluoropropyl dimethicone, and silicone elastomers, for instance the products sold under the KSG names by the company Shin-Etsu, under the name Trefil by the company Dow Corning or under the Gransil names by the company Grant Industries; and mixtures thereof.

These fatty substances mentioned above can be chosen in a varied way by the person skilled in the art so as to prepare a composition having the desired properties, for example of consistency or texture.

For emulsions, the proportion of fatty phase will be chosen according to the type of emulsion.

According to one embodiment, a composition according to the invention does not contain any silicone-based fatty substance.

The fatty phase may thus be present in the composition in an amount ranging from 1% to 80%, better still ranging from 5% to 70% and even better still from 10% to 60% by weight relative to the total weight of the composition.

Additives

A composition according to the invention may also comprise one or more additional agents chosen from antioxidants, plasticizers, coalescers, preserving agents, thickeners, fragrances, neutralizers, spreading agents, antifoams, dispersants and stabilizers, in particular chosen from surfactants and/or gelling agents, which are in particular hydrophilic, semicrystalline polymers, sweeteners, vitamins, trace elements, free-radical scavengers, sequestrants, demulcents, humectants other than the polyols and polyol derivatives suitable for use in the invention, opacifiers, emollients, silicones, fillers other than a synthetic phyllosilicate according to the invention, polymers, propellants, acidifying or basifying agents or any other ingredient normally used in the cosmetic and/or dermatological field, and mixtures thereof.

Similarly, a composition according to the invention may also comprise at least one dyestuff chosen, for example, from pigments, nacres, dyes and materials with an effect, and mixtures thereof.

These dyestuffs may be present in a content ranging from 0.01% to 50% by weight and preferably from 0.01% to 30% by weight relative to the total weight of the composition.

Likewise a composition according to the invention may also comprise at least one active agent chosen from moisturizers, cicatrizing agents and/or anti-aging agents for the skin, such as bodily and/or facial skin, and/or the nails.

Needless to say, all the abovementioned additional agents or compounds are different from the synthetic phyllosilicates described previously.

Needless to say, those skilled in the art will take care to select the optional additional ingredients or compounds and/or the amount thereof, in particular among those mentioned above, for the purpose of the intended use, but also such that the advantageous properties of the composition according to the invention are not, or are not substantially, adversely affected by the envisaged addition.

The additives are generally present in the composition according to the invention in an amount ranging from 0 to 20% by weight relative to the total weight of the composition.

Composition

The compositions according to the invention may be prepared according to the techniques that are well known to those skilled in the art.

The composition according to the invention may be in any presentation form conventionally used for the intended applications.

For example, when the composition according to the invention is cosmetic or dermatological, it may be in any presentation form conventionally used for topical applications and in particular in the form of a dispersion of aqueous gel or lotion type, of an emulsion of liquid to semi-solid consistency, obtained by dispersing a fatty phase in an aqueous phase (O/W) or conversely (W/O), or of a liquid to semi-solid suspension of emulsified gel or cream type.

Preferably, the composition is in the form of an oil-in-water emulsion (direct emulsion (O/W)) or water-in-oil emulsion (invert emulsion (W/O)), of a gel or of an emulsified gel, and even more preferentially in the form of a direct O/W or invert W/O emulsion. The emulsions may contain stabilizers, for instance fillers other than a synthetic phyllosilicate that is suitable for use in the invention, gelling polymers or thickening polymers.

According to one preferred embodiment, the composition according to the invention comprises a synthetic phyllosilicate, preferably in the form of an aqueous gel or of an aqueous-alcoholic gel, and at least one water-soluble organic UV-screening agent.

Such a combination makes it possible to obtain, after application to the skin, a notable improvement in the pleasantness, which is reflected in particular by a significant reduction in the tacky effect, by a sensation of freshness, a sensation of softness to the touch, a noticeable decrease in the greasiness and/or an improvement in the stability of the composition.

This combination makes it possible in particular to obtain a rheological effect insofar as there is a synergy between the gel and the sunscreen(s) used. Consequently, this results in an increased stability of the composition according to the invention.

According to one preferred embodiment, the composition according to the invention comprises a synthetic phyllosilicate, preferably in powder form, and at least one UV-screening agent, preferably chosen from liposoluble organic UV-screening agents, water-soluble organic screening agents, and mixtures thereof.

Such a combination makes it possible to obtain, after application to the skin, a notable improvement in the pleasantness, which is reflected in particular by a significant reduction in the tacky effect, a noticeable decrease in the greasy effect, a sensation of softness to the touch and/or an increase in the UV-radiation-screening efficiency, in particular in terms of the SPF, as demonstrated in the examples hereinafter.

This combination makes it possible to obtain a rheological effect insofar as there is a synergy between the gel and the sunscreen(s) used. Consequently, this results in an increased stability of the composition according to the invention.

As mentioned above, when a synthetic phyllosilicate that is suitable for use in the invention is in the form of a gel and more particularly of an aqueous or aqueous-alcoholic gel, it may constitute only part but also all of the aqueous phase. The aqueous phase may then optionally comprise, in addition to a synthetic phyllosilicate in gel form, one or more other gelling agents.

It should be noted that, additionally, these compositions defined in the above two paragraphs can also comprise a synthetic phyllosilicate in powder form in order to boost the sensory and/or screening performance level.

The cosmetic compositions according to the invention may be used, for example, as makeup products. More specifically, the makeup products may be of the type such as foundations, face powders, eyeshadows, concealer products or blushers, or alternatively a body makeup product or a skin and/or nail coloring product.

The cosmetic compositions according to the invention may be used, for example, as care products and/or anti-sun products for the face and/or body and/or nails with a liquid to semi-liquid consistency, such as milks, more or less smooth creams, cream gels or pastes.

They may optionally be packaged in aerosol form and may be in the form of a mousse or a spray.

The compositions according to the invention in the form of vaporizable fluid lotions in accordance with the invention are applied to the skin and/or the nails in the form of fine particles by means of pressurizing devices.

The devices suitable for use in the invention are well known to those skilled in the art and comprise non-aerosol pumps or “atomizers”, aerosol containers comprising a propellant and aerosol pumps using compressed air as propellant. These devices are described in U.S. Pat. No. 4,077,441 and U.S. Pat. No. 4,850,517.

The compositions packaged in aerosol form in accordance with the invention generally contain conventional propellants, for instance hydrofluoro compounds, dichlorodifluoromethane, difluoroethane, dimethyl ether, isobutane, n-butane, propane or trichlorofluoromethane. They are preferably present in amounts ranging from 15% to 50% by weight relative to the total weight of the composition.

Thus, for example, a composition according to the invention may in particular constitute a fragrancing composition, a deodorant composition or a composition for caring for and/or treating the skin and/or the nails and may in particular be in the form of a spray or an aerosol (body mist or body splash), an eau fraîche, an eau de toilette, an eau de parfum or an aftershave lotion.

Throughout the description, including the claims, the term “comprising a” should be understood as being synonymous with “comprising at least one”, unless otherwise specified.

The terms “between . . . and . . . ” and “ranging from . . . to . . . ” should be understood as being inclusive of the limits, unless otherwise specified.

In the description and the examples, the percentages are percentages by weight. The ingredients are mixed in the order and under the conditions that are easily determined by those skilled in the art.

### EXAMPLES

### Example 1: Preparation of a Synthetic Phyllosilicate that is Suitable for Use in the Invention

A synthetic phyllosilicate that is suitable for use in the invention is prepared according to the technology described in example 1 of application FR 2 977 580 from page 21, line 26 to page 23, line 20.

In the event where the phyllosilicate is in the form of an aqueous gel, the process was followed until the hydrogel formation without the drying step by lyophilization (from page 21 line 26 to page 22 line 29 of document FR 2 977 580).

Analysis of the x-ray diffractogram was performed with the aid of the materials and method used for the x-ray diffraction analyses that are detailed in application FR 2 977 580.

A characteristic diffraction line at 9.77 Å is observed.

The compositions according to the invention illustrated in the examples that follow comprise a synthetic phyllosilicate in accordance with the invention as obtained in this example 1.

### Example 2: Compositions and Evaluations of the Cosmetic Properties

Evaluation of the Cosmetic Properties

For each of the compositions A, B and/or C defined hereinafter, the cosmetic properties were evaluated according to the following protocol. The cosmetic properties on application are evaluated, monadically, by a panel of experts trained in the description of care products. The sensory evaluation of the care products by this panel is performed as follows: the products are packaged in opaque jars or pump-dispenser bottles depending on the viscosity of the products. Within the same session, the samples are presented in random order to each panellist.

The 15 Experts Evaluated the Following Parameters:
The final “tackiness” of the skin, after penetration.
The penetration sensation. This sensation is opposite to the sensation of formulae which “slip” during application, that is to say a formula which remains at the surface.

The descriptors are evaluated on a 3-level scale: +, ++ and +++.

For the “tackiness” parameter: + signifies Very tacky; ++ signifies Medium tackiness or Not Very tacky; and +++ signifies Not tacky.

For the “penetration sensation” parameter: + signifies No penetration sensation; ++ signifies Medium or Little penetration sensation; and +++ signifies Great deal of penetration sensation.

2.1 Creams of O/W Direct Emulsion Type Based on a Synthetic Phyllosilicate in Powder Form

Procedure:

Once the preserving system and the glycerine have been dissolved in water (at the necessary temperature), add the hydrophilic gelling agent with stirring (Rayneri deflocculator) at approximately 70° C. until homogenization of the gel. Homogenize the fatty phase (at the temperature necessary to have a homogeneous liquid phase). When the mixtures of the two phases are homogeneous, form the emulsion conventionally by adding the fatty phase to the aqueous phase with rotor/stator (Moritz) or mixer stirring. Cool with stirring (Rayneri deflocculator) and add the filler and the alcohol until a homogeneous smooth cream is obtained.

| | | COMPO- | COMPO- |
|---|---|---|---|
| | COMPO- | SITION B | SITION C |
| | SITION A | according to | according to |
| INCI Name/Ingredients | PLACEBO | the invention | the invention |
| HYDROGENATED | 10.0 | 10.0 | 10.0 |
| POLYISOBUTENE | | | |
| PARLEAM ® | | | |
| from the company NOF | | | |
| CORPORATION | | | |
| ARACHIDYL ALCOHOL | 2.1 | 2.1 | 2.1 |
| (and) BEHENYL ALCOHOL | | | |
| (and) ARACHIDYL | | | |
| GLUCOSIDE | | | |
| MONTANOV 202 ® | | | |
| from the company SEPPIC | | | |
| WATER | qs | qs | qs |
| GLYCERINE | 5.0 | 5.0 | 5.0 |
| PRESERVING AGENT | qs | qs | qs |
| SODIUM | 1.2 | 1.2 | 1.2 |
| ACRYLATE/SODIUM | | | |
| ACRYLOYLDIMETHYL | | | |
| TAURATE COPOLYMER | | | |
| (and) ISOHEXADECANE | | | |
| (and) POLYSORBATE 80 | | | |
| SIMULGEL EG ® | | | |
| from the company SEPPIC | | | |
| DENATURED ALCOHOL | 4.0 | 4.0 | 4.0 |
| Synthetic phyllosilicate | — | 2 | 5 |
| (POWDER) | | | |

Results

| | | COMPO- | COMPO- |
|---|---|---|---|
| | COMPO- | SITION B | SITION C |
| | SITION A | according to | according to |
| | PLACEBO | the invention | the invention |
| Tacky | + | ++ | ++ |
| effect | | | |
| Penetration | ++ | ++ (result > | +++ (result > |
| sensation | | than that | than that |
| | | of A) | of B) |

Thus, the introduction of a synthetic phyllosilicate suitable for use in the invention, in this case in powder form, to compositions B and C according to the invention makes it possible to decrease the sensation of tackiness of the emulsion and to increase the sensation of penetration of the composition during application.

The higher the amount of synthetic phyllosilicate suitable for use in the invention in the compositions, the more marked are these effects.

2.2 Creams of O/W Direct Emulsion Type Based on a Synthetic Phyllosilicate in Powder Form

Procedure:

Once the preserving system and the glycerine have been dissolved in water (at the necessary temperature), add the hydrophilic gelling agent with stirring (Rayneri deflocculator) at approximately 70° C. until homogenization of the gel. Homogenize the fatty phase (at the temperature necessary to have a homogeneous liquid phase). When the mixtures of the two phases are homogeneous, form the emulsion conventionally by adding the fatty phase to the aqueous phase with rotor/stator (Moritz) or mixer stirring. Cool with stirring (Rayneri deflocculator) and add the filler until a homogeneous smooth cream is obtained.

| | | COMPO- | COMPO- |
|---|---|---|---|
| | COMPO- | SITION B | SITION C |
| | SITION A | according to | according to |
| INCI Name/Ingredients | PLACEBO | the invention | the invention |
| HYDROGENATED | 12.0 | 12.0 | 12.0 |
| POLYISOBUTENE | | | |
| PARLEAM ® | | | |
| from the company NOF | | | |
| CORPORATION | | | |
| SUCROSE STEARATE | 1.5 | 1.5 | 1.5 |
| TEGOSOFT PSE 141 G ® | | | |
| from the company EVONIK | | | |
| GOLDSCHMIDT | | | |
| STEARIC ACID | 1.5 | 1.5 | 1.5 |
| WATER | qs | qs | qs |
| GLYCERINE | 5 | 5 | 5 |
| PRESERVING AGENT | qs | qs | qs |
| SODIUM | 1.2 | 1.2 | 1.2 |
| ACRYLATE/SODIUM | | | |
| ACRYLOYLDIMETHYL | | | |
| TAURATE COPOLYMER | | | |
| (and) ISOHEXADECANE | | | |
| (and) POLYSORBATE 80 | | | |
| SIMULGEL EG ® | | | |
| from the company SEPPIC | | | |
| Synthetic phyllosilicate | — | 2 | 5 |
| (POWDER) | | | |

Results

| | | COMPO- | COMPO- |
|---|---|---|---|
| | COMPO- | SITION B | SITION C |
| | SITION A | according to | according to |
| | PLACEBO | the invention | the invention |
| Tacky | + | ++ (result > | ++ (result > |
| effect | | than that | than that |
| | | of A) | of B) |
| Penetration | ++ | +++ (result > | +++ (result > |
| sensation | | than that | than that |
| | | of A) | of B) |

Thus, the introduction of a synthetic phyllosilicate suitable for use in the invention, in this case in powder form, to compositions B and C according to the invention makes it possible to decrease the sensation of tackiness of the emulsion and to increase the sensation of penetration of the composition during application.

The higher the amount of synthetic phyllosilicate suitable for use in the invention in the compositions, the more marked are these effects.

2.3 Creams of the Type (O/W Emulsion) Based on a Synthetic Phyllosilicate in Powder Form

Procedure:

Once the preserving system and the glycerine have been dissolved in water (at the necessary temperature), add the hydrophilic gelling agent with stirring (Rayneri deflocculator) at approximately 70° C. until homogenization of the gel. Homogenize the fatty phase (at the temperature necessary to have a homogeneous liquid phase). When the mixtures of the two phases are homogeneous, form the emulsion conventionally by adding the fatty phase to the aqueous phase with rotor/stator (Moritz) or mixer stirring. Cool with stirring (Rayneri deflocculator) and add the filler and the rhamnose until a homogeneous smooth cream is obtained.

| | | COMPO- | COMPO- |
|---|---|---|---|
| | COMPO- | SITION B | SITION C |
| | SITION a) | according to | according to |
| INCI Name/Ingredients | PLACEBO | the invention | the invention |
| GYCERYL STEARATE | 1.5 | 1.5 | 1.5 |
| (and) | | | |
| PEG-100 STEARATE | | | |
| ARLACEL 165-FL-(CQ) ® | | | |
| from the company CRODA | | | |
| HYDROGENATED | 12.0 | 12.0 | 12.0 |
| POLYISOBUTENE | | | |
| PARLEAM ® | | | |
| from the company NOF | | | |
| CORPORATION | | | |
| GLYCERINE | 2.0 | 2.0 | 2.0 |
| PRESERVING AGENT | qs | qs | qs |
| WATER | qs | qs | qs |
| RHAMNOSE | 1.5% | 1.5% | 1.5% |
| L RHAMNOSE MC ® | | | |
| from the company DANISCO | | | |
| SODIUM | 1.0 | 1.0 | 1.0 |
| ACRYLATE/SODIUM | | | |
| ACRYLOYLDIMETHYL | | | |
| TAURATE COPOLYMER | | | |
| (and) ISOHEXADECANE | — | — | — |
| (and) | | | |
| POLYSORBATE 80 | | | |
| SIMULGEL EG ® | | | |
| from the company SEPPIC | | | |
| Synthetic phyllosilicate | — | 2.0 | 5.0 |
| (POWDER) | | | |

Results

| | | COMPO- | COMPO- |
|---|---|---|---|
| | COMPO- | SITION B | SITION C |
| | SITION A | according to | according to |
| | PLACEBO | the invention | the invention |
| Tacky | + | ++ (result > | ++ (result > |
| effect | | than that | than that |
| | | of A) | of B) |
| Penetration | ++ | ++ (result > | +++ (result > |
| sensation | | than that | than that |
| | | of A) | of B) |

Thus, the introduction of a synthetic phyllosilicate suitable for use in the invention, in this case in powder form, to compositions B and C according to the invention makes it possible to decrease the sensation of tackiness of the emulsion and to increase the sensation of penetration of the composition during application.

The higher the amount of synthetic phyllosilicate suitable for use in the invention in the compositions, the more marked are these effects.

2.4 Creams of the Type (Emulsion Type: O/W) Based on a Synthetic Phyllosilicate in Powder Form

Procedure:

Once the preserving system and the glycerine have been dissolved in water (at the necessary temperature), add the hydrophilic gelling agent with stirring (Rayneri deflocculator) at approximately 70° C. until homogenization of the gel. Homogenize the fatty phase (at the temperature necessary to have a homogeneous liquid phase). When the mixtures of the two phases are homogeneous, form the emulsion conventionally by adding the fatty phase to the aqueous phase with rotor/stator (Moritz) or mixer stirring. Cool with stirring (Rayneri deflocculator) and add the filler and the mannose until a homogeneous smooth cream is obtained.

| | | COMPO- | COMPO- |
|---|---|---|---|
| | COMPO- | SITION B | SITION C |
| | SITION A | According to | According to |
| INCI Name/Ingredients | PLACEBO | the invention | the invention |
| GLYCERYL STEARATE SE | 2.5 | 2.5 | 2.5 |
| TEGIN PELLETS ® | | | |
| from the company EVONIK | | | |
| GOLDSCHMIDT | | | |
| HYDROGENATED | 12.0 | 12.0 | 12.0 |
| POLYISOBUTENE | | | |
| PARLEAM ® | | | |
| from the company NOF | | | |
| CORPORATION | | | |
| GLYCERINE | 2.0 | 2.0 | 2.0 |
| PRESERVING AGENT | qs | qs | qs |
| WATER | qs | qs | qs |
| MANNOSE | 1.5% | 1.5% | 1.5% |
| D-MANNOSE CT ® | | | |
| from the company DANISCO | | | |
| SODIUM ACRYLATE/ | 2.0 | 2.0 | 2.0 |
| SODIUM | | | |
| ACRYLOYLDIMETHYL | | | |
| TAURATE | | | |
| COPOLYMER (and) | | | |
| ISOHEXADECANE (and) | | | |
| POLYSORBATE 80 | | | |
| SIMULGEL EG ® | | | |
| from the company SEPPIC | | | |
| Synthetic phyllosilicate | — | 2.0 | 5.0 |
| (POWDER) | | | |

Results

| | | COMPO- | COMPO- |
|---|---|---|---|
| | COMPO- | SITION B | SITION C |
| | SITION A | according to | according to |
| | PLACEBO | the invention | the invention |
| Tacky | + | ++ (result > | ++ (result > |
| effect | | than that | than that |
| | | of A) | of B) |
| Penetration | ++ | ++ (result > | +++ (result > |
| sensation | | than that | than that |
| | | of A) | of B) |

Thus, the introduction of a synthetic phyllosilicate suitable for use in the invention, in this case in powder form, into compositions B and C according to the invention makes it possible to decrease the sensation of tackiness of the emulsion and to increase the sensation of penetration of the composition during application.

The higher the amount of synthetic phyllosilicate suitable for use in the invention in the compositions, the more marked are these effects.

2.5 Creams of O/W Direct Emulsion Type Based on a Synthetic Phyllosilicate in Powder Form

Procedure:

Once the preserving system and the glycerine have been dissolved in water (at the necessary temperature), add the hydrophilic gelling agent with stirring (Rayneri deflocculator) at approximately 70° C. until homogenization of the gel. Homogenize the fatty phase (at the temperature necessary to have a homogeneous liquid phase). When the mixtures of the two phases are homogeneous, form the emulsion conventionally by adding the fatty phase to the aqueous phase with rotor/stator (Moritz) or mixer stirring. Cool with stirring (Rayneri deflocculator) and add the filler until a homogeneous smooth cream is obtained.

| | | | COMPO- |
|---|---|---|---|
| | | COMPO- | SITION B |
| | | SITION A | according to |
| Phase | INCI Name/Ingredients | PLACEBO | the invention |
| Aqueous | WATER | qs | qs |
| phase | GLYCERINE | 40.0 | 40.0 |
| | PRESERVING AGENT | qs | qs |
| | TETRASODIUM EDTA | 0.1 | 0.1 |
| | SODIUM POLYACRYLATE | 0.8 | 0.8 |
| | COSMEDIA SP ® | | |
| | from the company | | |
| | COGNIS (BASF) | | |
| Fatty | COCOA BEAN BUTTER | 15.0 | 15.0 |
| phase | THEOBROMA | | |
| | CT COCOA BUTTER | | |
| | DEODORIZED ® | | |
| | from the company | | |
| | DUTCH COCOA BV | | |
| | GLYCERYL STEARATE | 2.5 | 2.5 |
| | CITRATE | | |
| | (and) POLYGLYCERYL-3 | | |
| | STEARATE | | |
| | (and) HYDROGENATED | | |
| | LECITHIN | | |
| | HELIOFEEL ® | | |
| | from the company | | |
| | LUCAS MEYER | | |
| | COSMETICS (UNIPEX) | | |
| filler | Synthetic phyllosilicate | — | 3.0 |
| | (POWDER) | | |

Results

| | | COMPO- |
|---|---|---|
| | COMPO- | SITION B |
| | SITION A | according to |
| | PLACEBO | the invention |
| Tacky | + | ++ |
| effect | | |

Thus, the introduction of a synthetic phyllosilicate suitable for use in the invention, in this case in powder form, into composition B according to the invention makes it possible to decrease the sensation of tackiness of the emulsion and to increase the sensation of penetration of the composition during application.

2.6 Creams of O/W Direct Emulsion Type Based on a Synthetic Phyllosilicate in Powder Form

Procedure:

Once the preserving system has been dissolved in water (at the required temperature), add the hydrophilic gelling agent with stirring (Rayneri deflocculator) at about 70° C. until the gel has homogenized, and add the active agent C-beta-D-xylopyranoside-2-hydroxy propane, the INCI name of which is Hydroxypropyl Tetrahydropyrantriol. Homogenize the fatty phase (at the temperature necessary to have a homogeneous liquid phase). When the mixtures of the two phases are homogeneous, form the emulsion by adding the fatty phase to the aqueous phase with rotor/stator (Moritz) or mixer stirring. Cool with stirring (Rayneri deflocculator) and add the filler until a homogeneous smooth cream is obtained.

| | | | | Composition B | Composition C |
|---|---|---|---|---|---|
| | | Composition A | | according to | according to |
| | INCI Name/Ingredients | PLACEBO | | the invention | the invention |
| Fatty | GLYCERYL STEARATE (and) | 2 | | 2 | 2 |
| phase | PEG-100 STEARATE | | | | |
| | ARLACEL 165-FL-(CQ) ® | | | | |
| | from the company CRODA | | | | |
| | HYDROGENATED | 10 | | 10 | 10 |
| | POLYISOBUTENE | | | | |
| | PARLEAM ® | | | | |
| | from the company NOF | | | | |
| | CORPORATION | | | | |
| Aqueous | PRESERVING AGENT | qs | | qs | qs |
| phase | WATER | qs | | qs | qs |
| | WATER/AQUA | 40% | 10 | 10 | 10 |
| | HYDROXYPROPYL | 35% | | | |
| | TETRAHYDROPYRANTRIOL/ | | | | |
| | HYDROXYPROPYL | | | | |
| | TETRAHYDROPYRANTRIOL | | | | |
| | PROPYLENE GLYCOL | 25% | | | |

| | | | | |
|---|---|---|---|---|
| | CETYL | 1 | 1 | 1 |
| | HYDROXYETHYLCELLULOSE | | | |
| | POLYSURF 67 CS ® | | | |
| | from the company ASHLAND | | | |
| Filler | Synthetic phyllosilicate (POWDER) | — | 2 | 5 |

The hydroxypropyl tetrahydropyrantriol composed of 40% of water, of 35% of hydroxypropyl tetrahydropyrantriol/hydroxypropyl tetrahydropyrantriol and of 25% of propylene glycol present in compositions A, B and C is sold under the name Mexoryl® by the company SBB Chimex.

Results

| | | COMPO- | COMPO- |
|---|---|---|---|
| | COMPO- | SITION B | SITION C |
| | SITION A | according to | according to |
| | PLACEBO | the invention | the invention |
| Tacky | + | ++ (result > | ++ (result > |
| effect | | than that | than that |
| | | of A) | of B) |
| Penetration | ++ | +++ (result > | +++ (result > |
| sensation | | than that | than that |
| | | of A) | of B) |

Thus, the introduction of a synthetic phyllosilicate suitable for use in the invention, in this case in powder form, into compositions B and C according to the invention makes it possible to decrease the sensation of tackiness of the emulsion and to increase the sensation of penetration of the composition during application.

The higher the amount of synthetic phyllosilicate suitable for use in the invention in the compositions, the more marked are these effects.

2.7 Cream of O/W Type Based on a Synthetic Phyllosilicate in Gel Form

Procedure:

Once the preserving system has been dissolved in water (at the required temperature), add the hydrophilic gelling agent with stirring (Rayneri deflocculator) at about 70° C. until the gel has homogenized, and add the active agent C-beta-D-xylopyranoside-2-hydroxy propane, the INCI name of which is Hydroxypropyl Tetrahydropyrantriol. Homogenize the fatty phase (at the temperature necessary to have a homogeneous liquid phase). When the mixtures of the two phases are homogeneous, form the emulsion by adding the fatty phase to the aqueous phase with rotor/stator (Moritz) or mixer stirring. Cool with stirring (Rayneri deflocculator) until a homogeneous smooth cream is obtained.

| | | | COMPO- |
|---|---|---|---|
| | | | SITION A |
| | | | according to |
| | INCI Name/Ingredients | | the invention |
| Fatty | GLYCERYL STEARATE (and) | | 2 |
| phase | PEG-100 STEARATE | | |
| | ARLACEL 165-FL-(CQ) ® | | |
| | from the company CRODA | | |
| | HYDROGENATED | | 12 |
| | POLYISOBUTENE | | |
| | PARLEAM ® | | |
| | from the company NOF | | |
| | CORPORATION | | |
| Aqueous | PRESERVING AGENT | | qs |
| phase | WATER | | qs |
| | WATER/AQUA | 40% | 10 |
| | HYDROXYPROPYL | 35% | |
| | TETRAHYDROPYRANTRIOL/ | | |
| | HYDROXYPROPYL | | |
| | TETRAHYDROPYRANTRIOL | | |
| | PROPYLENE GLYCOL | 25% | |

| | |
|---|---|
| Synthetic phyllosilicate (GEL) (10.3% am) | 30 |

The hydroxypropyl tetrahydropyrantriol composed of 40% of water, of 35% of hydroxypropyl tetrahydropyrantriol/hydroxypropyl tetrahydropyrantriol and of 25% of propylene glycol present in composition A is sold under the name Mexoryl® by the company SBB Chimex.

Thus, the introduction of a synthetic phyllosilicate suitable for use in the invention, in this case in gel form, into composition A according to the invention makes it possible to obtain a formula that exhibits very little tackiness and has a good penetration sensation.

2.8 Cream of O/W Emulsion Type Based on a Synthetic Phyllosilicate in Powder Form

| | | Composition A1 | Composition B1 | Composition C1 |
|---|---|---|---|---|
| | | (placebo) | (invention) | (invention) |
| | INGREDIENTS | Weight % | Weight % | Weight % |
| Fatty | DIISOPROPYL SEBACATE | 2.5 | 2.5 | 2.5 |
| phase | (DUB DIS ® - STEARINERIE DUBOIS) | | | |
| | SYNTHETIC WAX | 3 | 3 | 3 |
| | (CIREBELLE 303 ®- CIREBELLE) | | | |
| | ARACHIDYL ALCOHOL (and) | 2 | 2 | 2 |
| | BEHENYL ALCOHOL (and) | | | |
| | ARACHIDYL GLUCOSIDE | | | |
| | (MONTANOV 202 ® - SEPPIC) | | | |
| | OCTOCRYLENE | 7 | 7 | 7 |
| | (UVINUL N539 ® - BASF) | | | |
| | BUTYL | 3 | 3 | 3 |
| | METHOXYDIBENZOYLMETHANE | | | |
| | (PARSOL 1789 ® - DSM) | | | |
| | ETHYLHEXYL SALICYLATE | 5 | 5 | 5 |
| | (NEO HELIOPAN OS ®- SYMRISE) | | | |
| Aqueous | WATER | qs | qs | qs |
| phase | GLYCERINE | 5 | 5 | 5 |
| | PRESERVING AGENT | qs | qs | qs |
| | DISODIUM EDTA | 0.1 | 0.1 | 0.1 |
| | SODIUM ACRYLATE/SODIUM | 1.5 | 1.5 | 1.5 |
| | ACRYLOYLDIMETHYL TAURATE | | | |
| | COPOLYMER (and) | | | |
| | ISOHEXADECANE (and) | | | |
| | POLYSORBATE 80 | | | |
| | (SIMULGEL EG ® - SEPPIC) | | | |
| | DENATURED ALCOHOL | 4 | 4 | 4 |
| | SYNTHETIC PHYLLOSILICATE | — | 2 | 5 |
| | (POWDER) | | | |

Procedure:
Once the preserving system and the glycerine have been dissolved in water (at the necessary temperature), add the hydrophilic gelling agent with stirring using a Rayneri blender at approximately 70° C. until homogenization of the gel.
Homogenize the fatty phase (at the temperature necessary to have a homogeneous liquid phase).
When the mixtures of the two phases are homogeneous, form the emulsion conventionally by adding the fatty phase to the aqueous phase with stirring using a Moritz stirrer.
Cool with stirring using a Rayneri blender until a homogeneous smooth cream is obtained.
Add the filler(s) and the alcohol.

Compositions B and C according to the invention are stable.

Results—Evaluation of the Cosmetic Properties

For each of compositions A1, B1 and C1, the cosmetic properties were evaluated according to the following protocol.

The cosmetic properties on application are evaluated, monadically, by a panel of 15 experts trained in the description of care products.

The sensory evaluation of the care products by this panel is performed as follows: the products are packaged in opaque jars or pump-dispenser bottles depending on the viscosity of the products. Within the same session, the samples are presented in random order to each panellist.

The 15 experts evaluated the following parameters:
the final tackiness of the skin, after penetration;
the final greasy effect on the skin, after penetration;
the softness on application and finally. The “softness” descriptor is defined as the capacity of the product not to attach to the skin during application. The coarse effect or the dragging effect oppose this descriptor.

The descriptors are evaluated on a three-level scale: +, ++ and +++, the symbols + to +++ corresponding to an increase in the cosmetic property tested.

The results are collated in the table which follows.

| | Compo- | Compo- | Compo- |
|---|---|---|---|
| Parameters | sition A1 | sition B1 | sition C1 |
| evaluated | (placebo) | (invention) | (invention) |
| Tacky effect | +++ | ++ | + |
| Greasy effect | +++ | ++ | + |
| Softness | + | +++ | +++ |

The descriptors are evaluated on a three-level scale: +, ++ and +++, the symbols + to +++ corresponding to an increase in the cosmetic property tested.

Thus, the introduction of the synthetic phyllosilicate, in this case in powder form, into compositions B1 and C1 according to the invention containing liposoluble organic UV-screening agents makes it possible to reduce the tacky sensation and the greasy effect of the emulsions and also to improve the softness, that is to say to reduce the coarse/dragging effect.

Results—Evaluation of the SPF (Sun Protection Factor) In Vitro

In addition, an evaluation of the SPF (Sun Protection Factor) in vitro was carried out using a Labsphere® spectrophotometer.

The plate is the material to which the anti-sun composition is applied. For this protocol, poly(methyl methacrylate) (PMMA) plates proved to be ideal.

The results obtained are collated in the following table:

| | Compo- | Compo- | Compo- |
|---|---|---|---|
| | sition A1 | sition B1 | sition C1 |
| | (placebo) | (invention) | (invention) |
| SPF in-Vitro | 57.17 | 97.28 | 121.24 |
| Coefficient of variation | 19.00% | 17.10% | 28.90% |

Compositions B1 and C1 according to the invention comprising respectively 2% and 5% of synthetic phyllosilicate in powder form have a higher SPF factor than composition A1 not comprising synthetic phyllosilicate.

Thus, the presence of synthetic phyllosilicate introduced in powder form into the compositions containing lipophilic UV-screening agents makes it possible to increase the SPF performance level of the compositions according to the invention.

2.9 Cream of O/W Emulsion Type Based on a Synthetic Phyllosilicate in Powder Form

| | | Composition A2 | Composition B2 | Composition C2 |
|---|---|---|---|---|
| | | (placebo) | (invention) | (invention) |
| | INGREDIENTS | Weight % | Weight % | Weight % |
| Fatty | ISONONYL ISONONANOATE | 2 | 2 | 2 |
| phase | (DUB ININ ® - STEARINERIE | | | |
| | DUBOIS) | | | |
| | BEHENYL ALCOHOL (and) | 2 | 2 | 2 |
| | GLYCERYL STEARATE (and) | | | |
| | DISODIUM ETHYLENE | | | |
| | DICOCAMIDE PEG-15 | | | |
| | DISULFATE (and) GLYCERYL | | | |
| | STEARATE CITRATE | | | |
| | (CERALUTHION H ® - SASOL) | | | |
| | OCTOCRYLENE | 7 | 7 | 7 |
| | (UVINUL N539 ® - BASF) | | | |
| | BUTYL | 3 | 3 | 3 |
| | METHOXYDIBENZOYLMETHANE | | | |
| | (PARSOL 1789 ®- DSM) | | | |
| | ETHYLHEXYL SALICYLATE | 5 | 5 | 5 |
| | (NEO HELIOPAN OS ® - SYMRISE) | | | |
| Aqueous | WATER | qs | qs | qs |
| phase | GLYCERINE | 4 | 4 | 4 |
| | PRESERVING AGENT | qs | qs | qs |
| | DISODIUM EDTA | 0.1 | 0.1 | 0.1 |
| | SODIUM POLYACRYLATE | 1.5 | 1.5 | 1.5 |
| | (COSMEDIA SP ® - COGNIS) | | | |
| | SYNTHETIC PHYLLOSILICATE | — | 2 | 5 |
| | (POWDER) | | | |

Procedure:
Once the preserving system and the glycerine have been dissolved in water (at the necessary temperature), add the hydrophilic gelling agent with stirring using a Rayneri blender at approximately 70° C. until homogenization of the gel.
Homogenize the fatty phase (at the temperature necessary to have a homogeneous liquid phase).
When the mixtures of the two phases are homogeneous, form the emulsion conventionally by adding the fatty phase to the aqueous phase with stirring using a Moritz stirrer.
Cool with stirring using a Rayneri blender until a homogeneous smooth cream is obtained.
Add the filler(s).

Compositions B2 and C2 according to the invention are stable.

Results—Evaluation of the cosmetic properties

For each of compositions A2, B2 and C2, the cosmetic properties were evaluated according to the same protocol set out in example 2.1 above.

The results are collated in the table which follows.

| | Compo- | Compo- | Compo- |
|---|---|---|---|
| Parameters | sition A2 | sition B2 | sition C2 |
| evaluated | (placebo) | (invention) | (invention) |
| Tacky effect | +++ | ++ | + |
| Greasy effect | +++ | ++ | + |
| Softness | + | +++ | +++ |

The descriptors are evaluated on a three-level scale: +, ++ and +++, the symbols + to +++ corresponding to an increase in the cosmetic property tested.

Thus, the introduction of the synthetic phyllosilicate, in this case in powder form, into compositions B2 and C2 according to the invention containing lipophilic UV-screening agents makes it possible to reduce the tacky sensation and the greasy effect of the emulsions and also to improve the softness, that is to say to reduce the coarse/dragging effect.

The higher the amount of synthetic phyllosilicate introduced in powder form into the compositions according to the invention, the more marked are these effects.

Results—Evaluation of the SPF (Sun Protection Factor) In Vitro

In addition, an evaluation of the SPF (Sun Protection Factor) in vitro was carried out using a Labsphere® spectrophotometer on poly(methyl methacrylate) (PMMA) plates as described in detail in example 2.1 above.

The results obtained are collated in the following table:

| | Compo- | Compo- | Compo- |
|---|---|---|---|
| | sition A2 | sition B2 | sition C2 |
| | (placebo) | (invention) | (invention) |
| SPF in-Vitro | 78.33 | 108.8 | 105.33 |
| Coefficient of variation | 8.5% | 12.2% | 10.2% |

Compositions B2 and C2 according to the invention comprising respectively 2% and 5% of synthetic phyllosilicate in powder form have a higher SPF factor than composition A2 not comprising synthetic phyllosilicate.

2.10 Cream of O/W Emulsion Type Based on a Synthetic Phyllosilicate in Gel Form or in Gel and Powder Form

| | | Composition A3 | Composition B3 | Composition C3 |
|---|---|---|---|---|
| | | (invention) | (invention) | (placebo) |
| | INGREDIENTS | Weight % | Weight % | Weight % |
| Fatty | GLYCERYL STEARATE | 1.5 | 1.5 | 1.5 |
| phase | (TEGIN PELLETS ® - EVONIK | | | |
| | GOLDSCHMIDT) | | | |
| | ISOSTEARYL NEOPENTANOATE | 8 | 8 | 8 |
| | (DUB VCI 18 ®- STEARINERIE | | | |
| | DUBOIS) | | | |
| | ARACHIDYL ALCOHOL (and) | 1.5 | 1.5 | 1.5 |
| | BEHENYL ALCOHOL (and) | | | |
| | ARACHIDYL GLUCOSIDE | | | |
| | (MONTANOV 202 - SEPPIC) | | | |
| | (CERALUTHION H ® - SASOL) | | | |
| Aqueous | WATER | qs | qs | qs |
| phase | GLYCERINE | 3 | 3 | 3 |
| | PRESERVING AGENT | qs | qs | qs |
| | TRIETHANOLAMINE | 1.04 | 1.04 | 1.04 |
| | TROMETHAMINE | 2.52 | 2.52 | 2.52 |
| | (TRIS AMINO USP/EP GRADE ® - | | | |
| | ANGUS) | | | |
| | BIS-PEG-18 METHYL ETHER | 5 | 5 | 5 |
| | DIMETHYL SILANE | | | |
| | (DOW CORNING 2501 COSMETIC | | | |
| | WAX ® - DOW CORNING) | | | |
| | PHENYLBENZIMIDAZOLESULFONIC | 6 | 6 | 6 |
| | ACID | | | |
| | (EUSOLEX 232 ® - MERCK) | | | |
| | TEREPHTHALYLIDENE DICAMPHOR | 6 | 6 | 6 |
| | SULFONIC ACID | | | |
| | (MEXORYL SX ® - CHIMEX) | | | |
| | ACRYLATES/C10-30 ALKYL | — | — | 1 |
| | ACRYLATE CROSSPOLYMER | | | |
| | (CARBOPOL ULTREZ 20 | | | |
| | POLYMER ®-LUBRIZOL) | | | |
| | SYNTHETIC PHYLLOSILICATE | — | 5 | — |
| | (POWDER) | | | |
| | SYNTHETIC PHYLLOSILICATE (GEL) | 31 (3.19% am) | 31 (3.19% am) | — |
| | (10.3% am) | | | |
| | (am = active material) | | | |

Procedure:

It is identical to that described in detail in example 2.2.

Compositions A3, B3 and C3 according to the invention are stable.

Results—Evaluation of the Cosmetic Properties

For each of compositions A3, B3 and C3, the cosmetic properties were evaluated according to the same protocol set out in example 2.1 above.

The following additional parameter was also estimated among the 15 experts: freshness on application and after penetration.

The results are collated in the table which follows.

| | Compo- | Compo- | Compo- |
|---|---|---|---|
| Parameters | sition A3 | sition B3 | sition C3 |
| evaluated | (invention) | (invention) | (placebo) |
| Tacky effect | ++ | + | +++ |
| Greasy effect | ++ | + | +++ |
| Softness | +++ | +++ | + |
| Freshness | +++ | | + |

The descriptors are evaluated on a three-level scale: +, ++ and +++, the symbols + to +++ corresponding to an increase in the cosmetic property tested.

Thus, by comparing the results obtained for composition A3 according to the invention and for composition C3 (placebo, without synthetic phyllosilicate), it is demonstrated that the introduction of the synthetic phyllosilicate, in gel form and as a gelling agent, into a composition according to the invention containing water-soluble organic UV-screening agents makes it possible to reduce the tacky sensation and the greasy effect of the emulsions, to improve the softness, that is to say to reduce the coarse/dragging effect, and also to provide a lightness effect which goes as far as a fresh effect.

Composition B3 according to the invention comprising synthetic phyllosilicate incorporated both in powder form and in gel form provides great effects in terms of non-greasy effect and non-tacky effect.

### Example 3 Effect of the Synthetic Phyllosilicate Suitable for Use in the Invention on the Reduction of Tackiness

Procedure:

Once the preserving system and the glycerine have been dissolved in water (at the necessary temperature), add the hydrophilic gelling agent with stirring (Rayneri deflocculator) at approximately 70° C. until homogenization of the gel. Homogenize the fatty phase (at the temperature necessary to have a homogeneous liquid phase). When the mixtures of the two phases are homogeneous, form the emulsion conventionally by adding the fatty phase to the aqueous phase with stirring (Rayneri deflocculator) (or mixer). Cool with stirring (Rayneri deflocculator) until a homogeneous smooth cream is obtained.

| | | Compo- | Compo- | Compo- | Compo- |
|---|---|---|---|---|---|
| | | sition A | sition B | sition C | sition D |
| | | according to | outside the | outside the | outside the |
| | INCI Name/Ingredients | the invention | invention | invention | invention |
| Fatty | GLYCERYL | 2.0 | 2.0 | 2.0 | 2.0 |
| phase | STEARATE (and) | | | | |
| | PEG-100 STEARATE | | | | |
| | ARLACEL 165-FL- | | | | |
| | (CQ)- ® | | | | |
| | from the company | | | | |
| | CRODA | | | | |
| | ISOSTEARYL | 10.0 | 10.0 | 10.0 | 10.0 |
| | NEOPENTANOATE | | | | |
| | DUB VCI 18 ® | | | | |
| | from the company | | | | |
| | STEARINERIE | | | | |
| | DUBOIS | | | | |
| Aqueous | Phyllosilicate gel | 40.0 | — | 40.0 | — |
| phase | at 10.3% in water | | | | |
| | Preserving agent | qs | qs | qs | qs |
| | AMMONIUM | — | 1.5 | — | 1.5 |
| | POLYACRYLOYLDIMETHYL | | | | |
| | TAURATE | | | | |
| | HOSTACERIN | | | | |
| | AMPS ® | | | | |
| | from the company | | | | |
| | CLARIANT | | | | |
| | DIPROPYLENE | 10.0 | 10.0 | — | — |
| | GLYCOL | | | | |
| | PROPYLENE | 10.0 | 10.0 | — | — |
| | GLYCOL | | | | |
| | GLYCERINE | 10.0 | 10.0 | — | — |
| | WATER | qs | qs | qs | qs |

Results

| | Compo- | Compo- | Compo- | Compo- |
|---|---|---|---|---|
| | sition A | sition B | sition C | sition D |
| | according to | outside the | outside the | outside the |
| | the invention | invention | invention | invention |
| Tacky | +++ | + | +++ (result > | +++ (result > |
| effect | | | than that | than that |
| | | | of D) | of A) |
| Penetration | +++ (result > | ++ | +++ | +++ |
| sensation | than that | | | |
| | of D) | | | |

It proves to be the case that composition D outside the invention, without glycol, is not very tacky. On the other hand, when the composition comprises 30% of glycol (composition B outside the invention), it becomes very highly tacky. Thus, glycols induce a very strong tackiness sensation.

In addition, when comparing composition A according to the invention with composition B outside the invention, it appears that a synthetic phyllosilicate in gel form suitable for use in the invention makes it possible to reduce the tacky effect introduced by glycols compared with ammonium polyacryloyldimethyl taurate (AMPS).

Finally, the comparison between composition A according to the invention and composition C outside the invention makes it possible to demonstrate that the cosmetic properties of these two compositions are very similar, with or without a high amount of glycols (30% by weight).

These data demonstrate the capacity of a synthetic phyllosilicate suitable for use in the invention to reduce the tacky effect of a composition comprising a polyol.

1. A composition comprising:
(a) at least one synthetic phyllosilicate of molecular formula Mg₃Si₄O₁₀(OH)₂; and
(b) at least one polyol, and/or polyol derivative, and/or at least one UV-screening agent.

2. The composition as claimed in claim 1, wherein the composition has an X-ray diffraction line greater than 9.4 Å and less than or equal to 9.8 Å.

3. The composition as claimed in claim 1, wherein the composition has an infrared absorption band at 7200 cm⁻¹ corresponding to the stretching vibration attributed to the silanol groups Si—OH at the edge of the phyllosilicate leaflets.

4. The composition as claimed in claim 1, wherein there is no infrared absorption band at 7156 cm⁻¹.

5. The composition as claimed in claim 1, wherein said synthetic phyllosilicate is present in an amount ranging from 0.01% to 20% by weight relative to the total weight of the composition.

6. The composition as claimed in claim 1, wherein the synthetic phyllosilicate is in the form of an aqueous or aqueous-alcohol gel.

7. The composition as claimed in claim 1, wherein the synthetic phyllosilicate is in the form of an aqueous or aqueous-alcoholic gel and constitutes the aqueous phase.

8. The composition as claimed in claim 1, wherein the synthetic phyllosilicate in the form of an aqueous or aqueous-alcoholic gel is present in an amount ranging from 0.5% to 20% by weight of active material relative to the total weight of the aqueous phase.

9. The composition as claimed in claim 1, wherein the synthetic phyllosilicate is in powder form.

10. The composition as claimed in claim 1, wherein the synthetic phyllosilicate is in the form of an aqueous or aqueous-alcohol gel and in powder form.

11. The composition as claimed in claim 1, wherein the polyols are chosen from ethylene glycol, propylene glycol, 1,3-propanediol, isoprene glycol, butylene glycol, dipropylene glycol, polypropylene glycol, glycerol, glycerine, diglycerine, erythritol, pentaerithrytol, arabitol, adonitol, sorbitol, dulcitol, maltitol, panthenol, and also mixtures thereof.

12. The composition as claimed in claim 1, wherein the polyol is a sugar or a sugar derivative, and also mixtures thereof.

13. The composition as claimed in claim 1, wherein the sugar is a fatty acid ester of sugar chosen from: esters or mixtures of esters of C₈-C₂₂ fatty acid and of sucrose, maltose, glucose or fructose, and esters or mixtures of esters of C₁₄-C₂₂ fatty acid and of (C₁-C₄ alkyl) glucose, and mixtures thereof.

14. The composition as claimed in claim 1, wherein the polyol(s) are monosaccharides chosen from: trioses, tetroses, pentoses, hexoses, heptoses and octoses, and monosaccharides having more than 8 carbons.

15. The composition as claimed in claim 1, wherein the polyol is a sugar chosen from: Rhamnose, Mannose, Trehalose, Talose, Fucose, Ribose, Idose, Arabinose, Gulose, Xylose, Lyxose, Altrose, Allose, Glucose, Mannose, Galactose, Lactose, Sucrose, Cellobiose, Maltose, Fucose α(1-3) Glucose and Fructose, and also mixtures thereof.

16. The composition as claimed in claim 1, wherein the polyol and/or polyol derivative is present in a content of from 0.1% to 30% by weight relative to the total weight of the composition.

17. The composition as claimed in claim 1, wherein the UV-screening agent is chosen from water-soluble organic UV-screening agents, liposoluble organic UV-screening agents, insoluble organic UV-screening agents and mixtures thereof.

18. The composition as claimed in claim 17, wherein the water-soluble organic UV-screening agent is chosen from benzylidenecamphor compounds, phenyl benzimidazole compounds, and mixtures thereof.

19. The composition as claimed in claim 17, wherein the liposoluble organic UV-screening agent is chosen from dibenzoylmethane compounds, salicylic compounds, β,β-diphenylacrylate compounds, benzophenone compounds, phenyl benzotriazole compounds, triazine compounds and mixtures thereof.

20. The composition as claimed in claim 1, wherein the UV-screening agent(s) are present in amounts ranging from 0.1% to 40% by weight relative to the total weight of the composition.

21. The composition as claimed in claim 1, wherein the composition is cosmetic or dermatological and comprises a physiologically acceptable medium.

22. The composition as claimed in claim 1, wherein the synthetic phyllosilicate is in the form of an aqueous or aqueous-alcoholic gel and the UV-screening agent is a water-soluble organic UV-screening agent.

23. The composition as claimed in claim 1, said composition being in the form of a dispersion of aqueous gel or lotion type, of an emulsion with a liquid to semi-solid consistency, of direct O/W or invert W/O emulsions, or of a liquid to semi-solid suspension of emulsified gel or cream type.

24. The composition as claimed in claim 1, said composition being in the form of an emulsion, the fatty phase of which is present in the composition in an amount ranging from 1% to 80% by weight relative to the total weight of the composition.

25. A method for reducing the tacky effect of a composition comprising at least one polyol or a polyol derivative by using synthetic phyllosilicate of molecular formula Mg₃Si₄O₁₀(OH)₂ as defined in claim 1.

26. A method for increasing the moisturizing effect of a composition comprising at least one polyol or a polyol derivative by using synthetic phyllosilicate of molecular formula Mg₃Si₄O₁₀(OH)₂ as defined in claim 1.

27. A method for topically caring for the skin and/or the nails using the composition as claimed in claim 1.

28. (canceled)

29. A method for limiting the darkening of the skin and/or the nails and/or improving the color and/or uniformity of the complexion, comprising the application, to the surface of the skin and/or the nails, of at least one composition as defined in claim 1, said composition comprising at least one UV-screening agent.

30. A method for preventing and/or treating the signs of aging of the skin and/or of the nails, comprising the application, to the surface of the skin and/or the nails, of at least one composition as defined in claim 1, said composition comprising at least one UV-screening agent.
